(19) United States
(12) Patent Application Publication
(10) Pub. No.: US 2006/0058355 A1
(43) Pub. Date: Mar. 16, 2006

(54) Method of treatment of cardiac and/or renal failure using a calcium channel blocker and an angiotensin converting enzyme inhibitor or an angiotensin II receptor blocker

(76) Inventors: Howard C. Dittrich, San Diego, CA (US); Lauren Otsuki, San Diego, CA (US); Kenneth J. Widder, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/209,463

(22) Filed: Aug. 23, 2005

Related U.S. Application Data
(63) Continuation of application No. PCT/US04/05390, filed on Feb. 23, 2004.
(60) Provisional application No. 60/450,030, filed on Feb. 24, 2003.

(51) Int. Cl. A61K 31/455; A61K 31/4439

(52) U.S. Cl. 514/338; 514/355

(57) ABSTRACT

Disclosed is a pharmaceutical composition comprising a calcium channel blocker and an angiotensin II receptor blocker or an angiotensin converting enzyme inhibitor. Also disclosed is a method of treating cardiovascular disease or renal disease comprising identifying a patient in need of such treatment, and administering a pharmaceutical composition disclosed herein to said patient.

### RELATED APPLICATIONS

The present application is a continuation of PCT Patent Application Serial No. PCT/US2004/005390, filed Feb. 23, 2004 by Dittrich, et al. and entitled “METHOD OF TREATMENT OF CARDIAC AND/OR RENAL FAILURE USING A CALCIUM CHANNEL BLOCKER AND AN ANGIOTENSIN CONVERTING ENZYME INHIBITOR OR AN ANGIOTENSIN II RECEPTOR BLOCKER,” which in turn claims priority to U.S. Provisional Application Ser. No. 60/450,030, filed Feb. 24, 2003, by Dittrich, et al. and entitled “METHOD OF TREATMENT OF A DISEASE USING A CALCIUM CHANNEL BLOCKER AND AN ANGIOTENSIN CONVERTING ENZYME INHIBITOR OR AN ANGIOTENSIN II RECEPTOR BLOCKER,” all of which are incorporated by reference herein in their entirety, including any drawings.

### BACKGROUND OF THE INVENTION

1. Field of the Invention

The present invention relates to compositions comprising a combination of a calcium channel blocker and an Angiotensin II Receptor blocker (ARB) or an angiotensin converting enzyme (ACE) inhibitor and methods of treatment of patients suffering from cardiac and/or renal failure with said compositions.

2. Description of the Related Art

Calcium channel blockers block entry of calcium into cells, including the heart and arteries. Although the mechanism is complex, the result is that the contraction of the heart decreases and the arteries dilate. By dilating the arteries, the calcium channel blockers reduce arterial pressure. This makes it easier for the heart to pump blood and reduces the heart's oxygen requirement. Thus, calcium channel blockers are useful in the treatment of angina, a symptom caused by arteriosclerosis. Calcium channel blockers are also used to treat high blood pressure because of blood pressure lowering effects and abnormally rapid heart rhythms (e.g. atrial fibrillation) because they slow the heart rate. In addition, there is evidence that calcium channel blockers prevent kidney damage. Tzivoni, Dan, M.D. End organ protection by calcium-channel blockers” Clinical Cardiology 24, 102-106 (February 2001).

The pharmacological function and importance of calcium antagonists or calcium channel blockers, has been well documented. See, for example, R. A. Janis and D. J. Triggle “New developments in Ca²⁺ channel antagonists” Journal of Medicinal Chemistry, 26, 775-785 (1983). Among the calcium antagonists, 4-aryl-1,4-dihydropyridine-3,5-dicarboxylic diesters (DHPs) of the nifedipine type have become almost indispensable for the treatment of cardiovascular diseases. For a review on Structure Activity Relations (SAR) see, S. Goldmann and J. Stoltefuss “1,4-Dihydropyridine: Effects of chirality and conformation on the calcium antagonist and calcium agonist activities” Angewandte Chemie International Edition (English) 30, 1559-1578 (1991). It was well documented that substitution on 4-phenyl ring is very crucial for pharmacological activity. Substituents at ortho or meta position improve the activity, whereas para substitution invariably decrease the activity. It was also published that bulkiness of ortho substituent, improves the calcium antagonist activity. B. Loev, M. M Goodman, K. M. Snader, R. Tedeschi, E. Macko, “Hantzsch-Type Dihydropyridine hypotensive Agents”, Journal of Medicinal Chemistry 17, 956-965 (1974).

Voltage-gated calcium channels are large transmembrane proteins that regulate the intracellular concentration of calcium ions. They are classified into high (HVA) and low (LVA) voltage-activated channels according to the membrane potential at which they are activated. E. Carbone and H. D. Lux. “A low voltage activated, fully inactivating Ca channel in vertebrate sensory neurons” Nature, 310, 501-502, (1984): B. Nilius, P. Hess, J. B. Lansman and R. W. Tsien A novel type of cardiac calcium channel in ventricular cells. Nature, 316, 443-446. (1985).; M. C. Nowycky, A. P. Fox, R. W. Tsien. “Three types of neuronal calcium channels with different calcium agonist sensitivity” Nature 316, 440-443 (1985). LVA channels open and inactivate very fast, but deactivate about 10-100 times slower than HVA calcium channels. HVA channels require stronger membrane depolarizations to activate and can be divided further into N, P/Q, R and L-types based on their pharmacological properties. LVA channels can be detected in various tissues such as heart, brain, dorsal root ganglia and adrenal gland. The use of different search algorithms on mammalian expressed sequence tagged cDNAs or on similar sequences of the nematode Caenorhabditis elegans led to the identification of several genes, three of which encoded LVA calcium channels (T-type channels) and they have been named as α_{1G}, α_{1H}, α_{1I}; see Review, L. Lacinova, N. Klugbauer, F. Hofmann “Low voltage activated calcium channels: from genes to function” Gen. Physiol. Biophys., 19, 121-136, (2000). Of the above stated types of calcium channels, L-type channels received wide attention. Among the L-type channel blockers, Dihydropyridines (DHP) are the most widely studied. But, most of the DHPs are not selective against T-type channels and DHPs inhibiting the T-type channels are still sparse.

Voltage-gated calcium channels are important regulators of calcium influx in a number of cell types. Calcium entry through these channels activates a plethora of intracellular events, from the broad stimulation of gene expression, calcium-dependent second messenger cascades, and cell proliferation, to the specific release of neurotransmitter within the nervous system, and contraction in smooth and cardiac muscle (Tsien et al., 1988)(Wheeler et al., 1994); (Dunlap et al., 1995); (Tsien et al., 1991). A number of different types of calcium channels have been identified in native tissues and divided based on their biophysical profiles into low voltage activated (LVA) and high voltage activated (HVA) channels (Nowycky et al., 1985); (Tsien et al., 1991). LVA channels first activate at relatively hyperpolarized potentials and rapidly inactivate (Akaike et al., 1989); (Takahashi et al., 1991). By contrast, HVA channels require stronger membrane depolarizations to activate and can be divided further into N, P/Q-, R and L-types based on their pharmacological properties (for review, see (Stea et al., 1995); (Zamponi, 1997)). Molecular cloning has revealed that HVA channels are heteromultimers comprised of a pore forming α₁ subunit plus ancillary α₂-δ, β and possibly γ subunits (Pragnell et al., 1994); (Klugbauer et al., 1999); (Klugbauer et al., 2000); for review, see (Catterall, 2000), whereas LVA channels appear to contain only the α₁ subunit (Lacinova et al., 2000)). To date, ten different types of calcium channel α₁ subunits have been identified and shown to encode the previously identified native calcium channel isoforms. Expression studies show that alternative splicing of α_{1A} generates both P- and Q-type Ca²⁺ channels (Bourinet et al., 1999), α_{1B} encodes N-type channels (Dubel et al., 1992)) α_{1C}, α_{1D} and α_{1F} are L-type channels (Williams et al., 1992 b); (Bech-Hansen et al., 1998), α_{1G}, α_{1H} and α_{1I} form T-type channels (i.e., McRory et al., 2001) and α_{1E} may encode R-type channels (Soong et al., 1993); (Tottene et al., 1996), and α_{1S} encodes the skeletal muscle L-type channel isoform (Tanabe et al., 1987).

Dihydropyridine (DHP) antagonists of L-type calcium channels are widely used therapeutics in the treatment of hypertension, angina, arrhythmias, congestive heart failure, cardiomyopathy, arteriosclerosis, and cerebral and peripheral vascular disorders (Janis and Triggle, 1990) CRC Press, Cleveland. DHPs having a tendency to selectively block and enhance native L-type calcium channel activity. B. P. (Bean, 1984).; B. Z. (Peterson and Catterall, 1995). In addition to L-type channel activity, some of the DHPs are sensitive to T-type channel activity. (N. Akaike, H. Kanaide, T, Kuga, M, Nakamura, J. Sadoshima and Tomoike “Low Voltage Activated Calcium Current in rat Aorta Smooth Muscle Cells In Primary Culture” J Physiol. 416, 141-160, (1989).

Renin excreted from kidneys converts angiotensinogen produced in the liver to angiotensin I in the blood. Further, angiotensin I is converted to angiotensin II by angiotensin converting enzyme (ACE) in the lungs or plasma. The final active messenger of the renin-angiotensin pathway is angiotensin II. Angiotensin II binds to AT₁ receptors to cause vasoconstriction and fluid retention, both of which lead to an increase in blood pressure.

ACE inhibitors inhibit ACE in human subjects and animals. ACE is a peptidyl dipeptidase that catalyzes the conversion of angiotensin I to the vasoconstrictor substance, angiotensin II. Angiotensin II also stimulates aldosterone secretion by the adrenal cortex. Inhibition of ACE results in decreased plasma angiotensin II, which leads to decreased vasopressor activity and to decreased aldosterone secretion.

The angiotensin II receptor blockers lower blood pressure by blocking the AT₁ receptors. Therefore they have similar effects to angiotensin converting enzyme (ACE) inhibitors, which inhibit the synthesis of angiotensin II by ACE. However, non-ACE pathways can produce some angiotensin II. ACE inhibitors also decrease bradykinin breakdown and this action could be involved in some of the beneficial and adverse effects of that class of drugs.

### SUMMARY OF THE INVENTION

Disclosed is a pharmaceutical composition comprising a calcium channel blocker and an angiotensin II receptor blocker (ARB).

Also disclosed is a pharmaceutical composition comprising a calcium channel blocker and an angiotensin converting enzyme (ACE) inhibitor.

Also disclosed is a method of treating cardiovascular disease or renal disease comprising identifying a patient in need of such treatment, and administering a pharmaceutical composition disclosed herein to said patient.

### DETAILED DESCRIPTION OF THE PREFERRED EMBODIMENT

Aspects of the present invention relate to the treatment of renal and/or cardiovascular diseases using a combination of a calcium channel blocker (CCB), and an angiotensin converting enzyme (ACE) inhibitor or an angiotensin II receptor blocker (ARB). Each of these compounds have individually been shown to be somewhat effective in the treatment of cardiac disease, such as congestive heart failure, hypertension, asymptomatic left ventricular dysfunction, or acute myocardial infarction, or renal disease, such as diabetic nephropathy, contrast-mediated nephropathy, toxin-induced renal injury, or oxygen free-radical mediated nephropathy.

A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. The scope of the present invention includes all those ACE inhibitors now known and all those ACE inhibitors to be discovered in the future.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs' function stems from their ability to block the binding of angiotensin II, which would normally cause vessels to constrict.

The ACE inhibitors, ARBs, or CCBs used alone are typically insufficient to treat a given condition, such as hypertension or congestive heart failure (CHF). The combination of a T-type CCB with ACE inhibitor or ARB act synergistically, especially in high-resistant (non salt-sensitive) hypertensives.

In addition, given the lesser negative inotropic effect of T-type CCBs, the combinations of the present invention are more effective in patients with hypertension and left ventricular systolic (cardiac) dysfunction, since nonspecific or nonselective CCBs may make cardiac function worse. Likewise, the combinations of the present invention are more effective than any of the compounds used alone or a combination of nonselective CCBs with either ACE inhibitors or ARBs in CHF for the reasons set forth above.

In the treatment of renal disease, the combinations of the present invention are more effective than either any of the compounds used alone or a combination of nonselective CCBs with either ACE inhibitors or ARBs where there is renal vasoconstriction. The combinations of the present invention have a synergistic effect of dilating renal vasculature, thereby increasing the glomerular filtration rate (GFR), which in turn increases the loss of fluids.

Calcium channel blockers may decrease proteinuria and damage to kidney tissue. The present inventors have discovered that by combining an ARB with or without a diuretic in combination with a calcium channel blocker, multiple segments of the nephrons are targeted. As a result, patients in whom diuretics are becoming less effective will receive the benefit of the calcium channel blockers and the ARB. Further, the onset of lack of response to diuretics in those patients who are not refractory is delayed significantly.

Thus, in a first aspect, the invention relates to a pharmaceutical composition comprising an angiotensin converting enzyme (ACE) inhibitor and a T-type calcium channel blocker (CCB). The ACE inhibitor may be selected from the group consisting of lisinopril, enalapril, quinapril, ramipril, benazepril, captopril, fosinopril, moexipril, trandolapril, and perindopril, or a pharmaceutically acceptable salt, prodrug, ester, or amide thereof. However, the inclusion of other ACE inhibitors is within the scope of the present invention.

In another aspect, the invention relates to a pharmaceutical composition comprising an angiotensin II receptor blocker (ARB) and a T-type calcium channel blocker (CCB). The ARB may be selected from the group consisting of losartan, irbesartan, candesartan, telmisartan, eposartan, and valsartan, or a pharmaceutically acceptable salt, prodrug, ester, or amide thereof. However, the inclusion of other ARBs is within the scope of the present invention.

In a further aspect, the invention relates to a pharmaceutical composition comprising an ACE inhibitor, an ARB, and a T-type calcium channel blocker (CCB).

The T-type CCB of the invention may be a compound of Formula I or Formula II 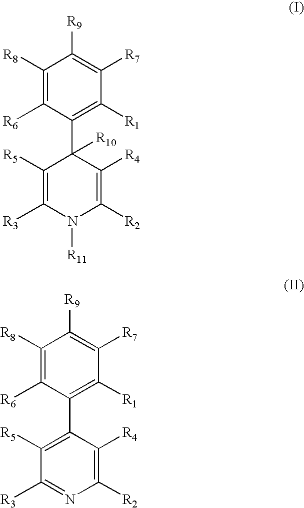
or a pharmaceutically acceptable salt, amide, ester, or prodrug thereof, where
a) R₁ is an straight-chain, branched, or cyclic alkyl group having greater than eight carbon atoms;
b) R₂-R₉ are each independently selected from the group consisting of hydrogen, halogen, perhaloalkyl, nitro, amino, a diazo salt, optionally substituted lower alkyl, optionally substituted lower alkylene, optionally substituted lower alkoxy, optionally substituted lower alkoxyalkyl, optionally substituted lower alkoxyalkoxy, optionally substituted lower mercaptyl, optionally substituted lower mercaptoalkyl, optionally substituted lower mercaptomercaptyl, —C(O)OH, —OC(O)H, —C(O)OR, —OC(O)R, —C(S)OR, —OC(S)R, —C(O)SR, —SC(O)R, —C(S)SR, —SC(S)R, C-amido, N-amido, and optionally substituted five- or six-membered heteroaryl ring or optionally substituted six-membered aryl or heteroaryl ring,
   where the lower alkyl and the lower alkylene moieties are each independently and optionally substituted with one or more substituents selected from the group consisting of halogen, perhaloalkyl, nitro, amino, hydroxy, alkoxy, sulfhydryl, thioether, cyano, amido, ester, and 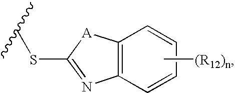
      where A is selected from the group consisting of oxygen, sulfur, and —NH and R₁₂ is selected for the group consisting of hydrogen, hydroxy, alkoxy, haloalkoxy, halogen, haloalkyl, perhaloalkyl, nitro, amino, and a diazo salt, and n is between 0-4; and
   where the ring moieties are each independently and optionally substituted with one or more substituents selected from the group consisting of lower alkyl, lower alkylene,
c) R₁₀ and R₁₁ in the compound of Formula I are each independently selected from the group consisting of hydrogen and lower alkyl.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are optionally substituted and are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are optionally substituted and are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

Any amine, hydroxy, or carboxyl side chain on the compounds of the present invention can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” refers to an aromatic group which contains at least one heterocyclic ring.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The alkyl group may have 1 to 40 carbon atoms (whenever it appears herein, a numerical range such as “1 to 40” refers to each integer in the given range; e.g., “1 to 40 carbon atoms” means that the alkyl group may consist of 1 carbon atom, 2 carbon atoms, 3 carbon atoms, etc., up to and including 40 carbon atoms, although the present definition also covers the occurrence of the term “alkyl” where no numerical range is designated). The alkyl group may also be a medium size alkyl having 1 to 20 carbon atoms. The alkyl group could also be a lower alkyl having 1 to 5 carbon atoms. The alkyl group of the compounds of the invention may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethly, propyl, iso-propyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substituent may be substituted with one of the above substituents.

The substituent “R” or “R′” appearing by itself and without a number designation refers to an optionally substituted substituent selected from the group consisting of alkyl, cycloalkyl, aryl, heteroaryl (bonded through a ring carbon) and heteroalicyclic (bonded through a ring carbon).

An “alkoxy” group refers to a RO— group, where R is as defined herein.

An “alkoxyalkyl” group refers to a R′OR— group, where R and R′ are as defined herein.

An “alkoxyalkoxy” group refers to a ROR′O— group, where R is as defined herein.

An “mercaptyl” group refers to a RS— group, where R is as defined herein.

An “mercaptoalkyl” group refers to a R′SR— group, where R and R′ are as defined herein.

An “mercaptomercaptyl” group refers to a RSR′S— group, where R is as defined herein.

An “O-carboxy” group refers to a RC(═O)O— group, where R is as defined herein.

A “C-carboxy” group refers to a —C(═O)OR groups where R is as defined herein.

An “acetyl” group refers to a —C(═O)CH₃, group.

A “trihalomethane sulfonyl” group refers to a X₃CS(═O)₂— group where X is a halogen.

A “cyano” group refers to a —CN group.

An “isocyanato” group refers to a —NCO group.

A “thiocyanato” group refers to a —CNS group.

An “isothiocyanato” group refers to a —NCS group.

A “sulfinyl” group refers to a —S(═O)—R group, with R as defined herein.

A “S-sulfonamido” group refers to a —S(═O)₂NR, group, with R as defined herein.

A “N-sulfonamido” group refers to a RS(═O)₂NH— group with R as defined herein.

A “trihalomethanesulfonamido” group refers to a X₃CS(═O)₂NR— group with X and R as defined herein.

An “O-carbamyl” group refers to a —OC(═O)—NR, group-with R as defined herein.

An “N-carbamyl” group refers to a ROC(═O)NH— group, with R as defined herein.

An “O-thiocarbamyl” group refers to a —OC(═S)—NR, group with R as defined herein.

An “N-thiocarbamyl” group refers to an ROC(═S)NH— group, with R as defined herein.

A “C-amido” group refers to a —C(═O)—NR₂ group with R as defined herein.

An “N-amido” group refers to a RC(═O)NH— group, with R as defined herein.

The term “perhaloalkyl” refers to an alkyl group where all of the hydrogen atoms are replaced by halogen atoms.

Unless otherwise indicated, when a substituent is deemed to be “optionally subsituted,” it is meant that the subsitutent is a group that may be substituted with one or more group(s) individually and independently selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

In certain embodiments, in the compound of Formula I or II, R₁ is an optionally substituted alkyl group having greater than or equal to ten carbon atoms. In other embodiments, R₁ has greater than or equal to twelve carbon atoms, whereas in other embodiments, R₁ has greater than or equal to fifteen carbon atoms. In some embodiments, R₁ is a C₁₀ straight-chain alkyl group, or a C₁₁ straight-chain alkyl group, or a C₁₂ straight-chain alkyl group, or a C₁₃ straight-chain alkyl group, or a C₁₄ straight-chain alkyl group, or a C₁₅ straight-chain alkyl group. In certain embodiments

In certain embodiments, R₂ and R₃ are each independently an optionally substituted alkyl group. In some embodiments, R₂ and R₃ are the same, whereas in other embodiments, they are different. In certain embodiments, R₂ and R₃ are lower alkyl. In certain compounds of Formula I or II, R₂ and R₃ are each independently selected from methyl, ethyl, or isopropyl. Embodiments of the present invention include those in which R₂ and R₃ are the same and they both are methyl.

In certain embodiments, R₄ is 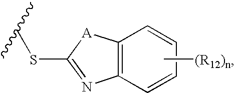
where A is selected from the group consisting of oxygen, sulfur, and —NH and R₁₂ is selected from the group consisting of hydrogen, hydroxy, alkoxy, haloalkoxy, halogen, haloalkyl, perhaloalkyl, nitro, amino, and a diazo salt, and n is between 0-4.

A “diazo salt” is a group of formula —NN⁺X⁻, where X is a halogen. In some embodiments, the halogen is a chlorine, while in other embodiments, the halogen is a fluorine, or a bromine.

In some embodiments A is oxygen, while in other embodiments A is sulfur, and in still other embodiments A is —NH.

R₄ and R₅ may be the same or different. In some embodiments, R₄ and R₅ are selected from the group consisting of
a) an optionally substituted alkyl group;
b) an alkoxy of formula —(X₁)ₙ₁—O—X₂, where
   X₁ is selected from the group consisting of lower alkylene, lower alkenylene, lower alkynylene, aryl, and heteroaryl;
   X₂ is selected from the group consisting of hydrogen, lower alkyl, aryl, and heteroaryl; and
   n1 is 0 or 1; and
c) a thioether or thiol of formula —(X₃)ₙ₃—S—X₄, where
   X₃ is selected from the group consisting of lower alkylene, lower alkenylene, lower alkynylene, aryl, and heteroaryl;
   X₄ is selected from the group consisting of hydrogen, lower alkyl, aryl, and heteroaryl; and
   n3 is 0 or 1;
d) a carboxylic acid of formula —(X₅)ₙ₅—C(=E)-E′H, where
   X₅ is selected from the group consisting of lower alkylene, lower alkenylene, lower alkynylene, aryl, and heteroaryl;
   E and E′ are each independently selected from the group consisting of oxygen and sulfur;
   n5 is 0 or 1; and
e) an ester of formula —(X₆)ₙ₆—C(=E)-E′X₇, or of formula —(X₆)ₙ₆-E′-C(=E)-X₇, where
   X₆ is selected from the group consisting of lower alkylene, lower alkenylene, lower alkynylene, aryl, and heteroaryl;
   E and E′ are each independently selected from the group consisting of oxygen and sulfur;
   X₇ is selected from the group consisting of hydrogen, lower alkyl, aryl, heteroaryl, hydroxy, alkoxy, amino, and —NX₈X₉,
      where X₈ and X₉ are each independently selected from the group consisting of hydrogen, alkyl, aryl, and heteroaryl; and
   n6 is 0 or 1.

In some embodiments, R₄ and R₅ are each independently lower alkyl. In certain embodiments, R₄ and R₅ are selected from the group consisting of methyl, ethyl, n-propyl, isopropyl, n-butyl, sec-butyl, and tert-butyl.

In other embodiments, where n1, n3, n5, or n6 in the above formulae is each independently 1, then X₁, X₃, X₅, and X₆ are each independently methylene (—CH₂—). In certain embodiments, X₂, X₄, and X₇ are each independently lower alkyl. The lower alkyl may be selected from the group consisting of methyl, ethyl, and isopropyl.

In certain embodiments, E and E′ are each independently oxygen, whereas in other embodiments E may be sulfur and E′, if it exists, oxygen.

In certain embodiments, R₄ and R₅ are each independently selected from the group consisting of —C(O)OH, —C(O)OCH₃, —C(O)OCH₂CH₃, —C(O)OCH(CH₃)₂, —CH₂OCH₃, —CH₂OCH₂CH₃, and —CH₂OCH(CH₃)₂.

In certain embodiments, R₆ is selected from the group consisting of
a) hydrogen;
b) an optionally substituted alkyl group;
c) an alkoxy of formula —(X₁)ₙ₁—O—X₂, where
   X₁ is selected from the group consisting of lower alkylene, lower alkenylene, lower alkynylene, aryl, and heteroaryl;
   X₂ is selected from the group consisting of hydrogen, lower alkyl, aryl, and heteroaryl; and
   n1 is 0 or 1; and
d) a thioether or thiol of formula —(X₃)ₙ₃—S—X₄, where
   X₃ is selected from the group consisting of lower alkylene, lower alkenylene, lower alkynylene, aryl, and heteroaryl;
   X₄ is selected from the group consisting of hydrogen, lower alkyl, aryl, and heteroaryl; and
   n3 is 0 or 1;
e) a carboxylic acid of formula —(X₅)ₙ₅—C(=E)-E′H, where
   X₅ is selected from the group consisting of lower alkylene, lower alkenylene, lower alkynylene, aryl, and heteroaryl;
   E and E′ are each independently selected from the group consisting of oxygen and sulfur;
   n5 is 0 or 1; and
f) an ester of formula —(X₆)ₙ₆—C(=E)-E′X₇, or of formula —(X₆)ₙ₆-E′-C(=E)-X₇, where
   X₆ is selected from the group consisting of lower alkylene, lower alkenylene, lower alkynylene, aryl, and heteroaryl;
   E and E′ are each independently selected from the group consisting of oxygen and sulfur;
   X₇ is selected from the group consisting of hydrogen, lower alkyl, aryl, heteroaryl, hydroxy, alkoxy, amino, and —NX₈X₉,
      where X₈ and X₉ are each independently selected from the group consisting of hydrogen, alkyl, aryl, and heteroaryl; and
   n6 is 0 or 1.

In certain embodiments, the alkyl mentioned above is a lower alkyl. In some of these embodiments, the alkyl is selected from the group consisting of methyl, ethyl, and isopropyl. In certain other embodiments, R₆ is an alkoxy selected from the group consisting of methoxy, ethoxy, and isopropoxy.

In certain embodiments R₇-R₉ are each independently selected from the group consisting of
a) hydrogen;
b) an optionally substituted alkyl group;
c) an alkoxy of formula —(X₁)ₙ₁—O—X₂, where
   X₁ is selected from the group consisting of lower alkylene, lower alkenylene, lower alkynylene, aryl, and heteroaryl;
   X₂ is selected from the group consisting of hydrogen, lower alkyl, aryl, and heteroaryl; and
   n1 is 0 or 1; and
d) a thioether or thiol of formula —(X₃)ₙ₃—S—X₄, where
   X₃ is selected from the group consisting of lower alkylene, lower alkenylene, lower alkynylene, aryl, and heteroaryl;
   X₄ is selected from the group consisting of hydrogen, lower alkyl, aryl, and heteroaryl; and
   n3 is 0 or 1;
e) a carboxylic acid of formula —(X₅)ₙ₅—C(=E)-E′H, where
   X₅ is selected from the group consisting of lower alkylene, lower alkenylene, lower alkynylene, aryl, and heteroaryl;
   E and E′ are each independently selected from the group consisting of oxygen and sulfur;
   n5 is 0 or 1;
f) an ester of formula —(X₆)ₙ₆—C(=E)-E′X₇, or of formula —(X₆)ₙ₆-E′-C(=E)-X₇, where
   X₆ is selected from the group consisting of lower alkylene, lower alkenylene, lower alkynylene, aryl, and heteroaryl;
   E and E′ are each independently selected from the group consisting of oxygen and sulfur;
   X₇ is selected from the group consisting of hydrogen, lower alkyl, aryl, heteroaryl, hydroxy, alkoxy, amino, and —NX₈X₉,
      where X₈ and X₉ are each independently selected from the group consisting of hydrogen, alkyl, aryl, and heteroaryl; and
   n6 is 0 or 1;
g) an amine of formula —(X₁₀)ₙ₁₀—NX₁₁X₁₂, where
   X₁₀ is selected from the group consisting of lower alkylene, lower alkenylene, lower alkynylene, aryl, and heteroaryl;
   where X₁₀ and X₁₁ are each independently selected from the group consisting of hydrogen, alkyl, aryl, and heteroaryl; and
n10 is 0 or 1;
h) NO₂;
i) halogen or perhaloalkyl; and
j) CN.

In certain embodiments, the alkyl mentioned above is a lower alkyl. In some of these embodiments, the alkyl is selected from the group consisting of methyl, ethyl, and isopropyl. In certain other embodiments, R₇-R₉ are each independently hydrogen, hydroxy, cyano (CN), nitro (NO₂), amino (NH₂), methyl, ethyl, isopropyl, fluoro, and chloro. It is understood that in some embodiments R₇-R₉ are the same, whereas in other embodiments, R₇-R₉ are different.

In certain embodiments R₁₀ and R₁₁ are each independently selected from the group consisting of hydrogen and alkyl. In certain embodiments, the alkyl is a lower alkyl. In some of these embodiments, the alkyl is selected from the group consisting of methyl, ethyl, and isopropyl.

The compounds of the present invention are shown here without designating any particular stereochemistry. Some of the compounds of the present invention posses a chiral center and exhibit optical isomerism. It is understood that the scope of the present invention includes a racemic mixture of the isomer, in addition to the individual S and R isomers of the compounds disclosed herein. Separation of optical isomers from a racemic mixture can be accomplished using methods known to those of ordinary skill in the art.

In certain embodiments, the present invention relates to a compound of Formula I or II, where the compound is selected from the group consisting of
diethyl 1,4-dihydro-4-(2′-ethoxy-6′-pentadecylphenyl)-2,6-dimethyl-3,5-pyridine dicarboxylate;
dimethyl 1,4-dihydro-4-(2′-ethoxy-6′-pentadecylphenyl)-2,6-dimethyl-3,5-pyridine dicarboxylate;
diisopropyl 1,4-dihydro-4-(2′-ethoxy-6′-pentadecylphenyl)-2,6-dimethyl-3,5-pyridine dicarboxylate;
diethyl 1,4-dihydro-4-(2′-methoxy-6′-pentadecylphenyl)-2,6-dimethyl-3,5-pyridine dicarboxylate;
dimethyl 1,4-dihydro-4-(2′-methoxy-6′-pentadecylphenyl)-2,6-dimethyl-3,5-pyridine dicarboxylate;
diisopropyl 1,4-dihydro-4-(2′-methoxy-6′-pentadecylphenyl)-2,6-dimethyl-3,5-carboxypyridine dicarboxylate;
diethyl 1,4-dihydro-4-(2′-isopropoxy-6′-pentadecylphenyl)-2,6-dimethyl-3,5-pyridine dicarboxylate;
dimethyl 1,4-dihydro-4-(2′-isopropoxy-6′-pentadecylphenyl)-2,6-dimethyl-3,5-pyridine dicarboxylate;
diisopropyl 1,4-dihydro-4-(2′-isopropoxy-6′-pentadecylphenyl)-2,6-dimethyl-3,5-pyridine dicarboxylate;
diethyl 1,4-dihydro-4-(2′-methoxy-6′-pentadecylphenyl)-2-methyl-6-(2′-mercapto-1′H-benzimidazolyl)methyl-3,5-pyridine dicarboxylate;
dimethyl 1,4-dihydro-4-(2′-methoxy-6′-pentadecylphenyl)-2-methyl-6-(2′-mercapto-1′H-benzimidazolyl)methyl-3,5-pyridine dicarboxylate;
diisopropyl 1,4-dihydro-4-(2′-methoxy-6′-pentadecylphenyl)-2-methyl-6-(2′-mercapto-1′H-benzimidazolyl)methyl-3,5-pyridine dicarboxylate;
diethyl 1,4-dihydro-4-(2′-isopropoxy-6′-pentadecylphenyl)-2-methyl-6-(2′-mercapto-1′H-benzimidazolyl)methyl-3,5-pyridine dicarboxylate;
dimethyl 1,4-dihydro-4-(2′-isopropoxy-6′-pentadecylphenyl)-2-methyl-6-(2′-mercapto-1′H-benzimidazolyl)methyl-3,5-pyridine dicarboxylate;
diisopropyl 1,4-dihydro-4-(2′-isopropoxy-6′-pentadecylphenyl)-2-methyl-6-(2′-mercapto-1′H-benzimidazolyl)methyl-3,5-pyridine dicarboxylate;
diethyl 1,4-dihydro-4-(2′-ethoxy-6′-pentadecylphenyl)-2-methyl-6-(2′-mercapto-1′H-benzimidazolyl)methyl-3,5-pyridine dicarboxylate;
dimethyl 1,4-dihydro-4-(2′-ethoxy-6′-pentadecylphenyl)-2-methyl-6-(2′-mercapto-1′H-benzimidazolyl)methyl-3,5-pyridine dicarboxylate;
diisopropyl 1,4-dihydro-4-(2′-ethoxy-6′-pentadecylphenyl)-2-methyl-6-(2′-mercapto-1′H-benzimidazolyl)methyl-3,5-pyridine dicarboxylate;
1,4-dihydro-4-(2′-ethoxy-6′-pentadecylphenyl)-2,6-dimethyl-3-ethyl-5-(methoxyethyl)pyridine dicarboxylate;
1,4-dihydro-4-(2′-ethoxy-6′-pentadecylphenyl)-2,6-dimethyl-3-methyl-5-(methoxyethyl)pyridine dicarboxylate;
1,4-dihydro-4-(2′-ethoxy-6′-pentadecylphenyl)-2,6-dimethyl-3-isopropyl-5-(methoxyethyl)pyridine dicarboxylate;
1,4-dihydro-4-(2′-methoxy-6′-pentadecylphenyl)-2,6-dimethyl-3-ethyl-5-(methoxyethyl)pyridine dicarboxylate;
1,4-dihydro-4-(2′-isopropoxy-6′-pentadecylphenyl)-2,6-dimethyl-3-ethyl-5-(methoxyethyl)pyridine dicarboxylate;
diethyl 1,4-dihydro-4-(2′-ethoxy-6′-pentadecylphenyl)-2-(2′-aminoethoxy)methyl-6-methyl-3,5-pyridine dicarboxylate;
dimethyl 1,4-dihydro-4-(2′-ethoxy-6′-pentadecylphenyl)-2-(2′-aminoethoxy)methyl-6-methyl-3,5-pyridine dicarboxylate;
diisopropyl 1,4-dihydro-4-(2′-ethoxy-6′-pentadecylphenyl)-2-(2′-aminoethoxy)methyl-6-methyl-3,5-pyridine dicarboxylate;
diethyl 1,4-dihydro-4-(2′-methoxy-6′-pentadecylphenyl)-2-(2′-aminoethoxy)methyl-6-methyl-3,5-pyridine dicarboxylate;
dimethyl 1,4-dihydro-4-(2′-methoxy-6′-pentadecylphenyl)-2-(2′-aminoethoxy)methyl-6-methyl-3,5-pyridine dicarboxylate;
diisopropyl 1,4-dihydro-4-(2′-methoxy-6′-pentadecylphenyl)-2-(2′-aminoethoxy)methyl-6-methyl-3,5-pyridine dicarboxylate;
diethyl 1,4-dihydro-4-(2′-isopropoxy-6′-pentadecylphenyl)-2-((2′-aminoethoxy)methyl-6-methyl-3,5-pyridine dicarboxylate;
dimethyl 1,4-dihydro-4-(2′-isopropoxy-6′-pentadecylphenyl)-2-(2′-aminoethoxy)methyl-6-methyl-3,5-pyridine dicarboxylate;
diisopropyl 1,4-dihydro-4-(2′-isopropoxy-6′-pentadecylphenyl)-2-(2′-aminoethoxy)methyl-6-methyl-3,5-pyridine dicarboxylate;
diethyl 1,4-dihydro-4-(2′-ethoxy-3′,5′-dinitro-6′-pentadecylphenyl)-2,6-dimethyl-3,5-pyridine dicarboxylate;
dimethyl 1,4-dihydro-4-(2′-ethoxy-3′,5′-dinitro-6′-pentadecylphenyl)-2,6-dimethyl-3,5-pyridine dicarboxylate;
diisopropyl 1,4-dihydro-4-(2′-ethoxy-3′,5′-dinitro-6′-pentadecylphenyl)-2,6-dimethyl-3,5-pyridine dicarboxylate;
diethyl 1,4-dihydro-4-(2′-methoxy-3′,5′-dinitro-6′-pentadecylphenyl)-2,6-dimethyl-3,5-pyridine dicarboxylate;
dimethyl 1,4-dihydro-4-(2′-methoxy-3′,5′-dinitro-6′-pentadecylphenyl)-2,6-dimethyl-3,5-pyridine dicarboxylate;
diisopropyl 1,4-dihydro-4-(2′-methoxy-3′,5′-dinitro-6′-pentadecylphenyl)-2,6-dimethyl-3,5-pyridine dicarboxylate;
diethyl 1,4-dihydro-4-(2′-isopropoxy-3′,5′-dinitro-6′-pentadecylphenyl)-2,6-dimethyl-3,5-pyridine dicarboxylate;
dimethyl 1,4-dihydro-4-(2′-isopropoxy-3′,5′-dinitro-6′-pentadecylphenyl)-2,6-dimethyl-3,5-pyridine dicarboxylate;
diisopropyl 1,4-dihydro-4-(2′-isopropoxy-3′,5′-dinitro-6′-pentadecylphenyl)-2,6-dimethyl-3,5-pyridine dicarboxylate;
diethyl 1,4-dihydro-4-(2′-ethoxy-3′,5′-diamino-6′-pentadecylphenyl)-2,6-dimethyl-3,5-pyridine dicarboxylate;
dimethyl 1,4-dihydro-4-(2′-ethoxy-3′,5′-diamino-6′-pentadecylphenyl)-2,6-dimethyl-3,5-pyridine dicarboxylate;
diisopropyl 1,4-dihydro-4-(2′-ethoxy-3′,5′-diamino-6′-pentadecylphenyl)-2,6-dimethyl-3,5-pyridine dicarboxylate;
diethyl 1,4-dihydro-4-(2′-methoxy-3′,5′-diamino-6′-pentadecylphenyl)-2,6-dimethyl-3,5-pyridine dicarboxylate;
dimethyl 1,4-dihydro-4-(2′-methoxy-3′,5′-diamino-6′-pentadecylphenyl)-2,6-dimethyl-3,5-pyridine dicarboxylate;
diisopropyl 1,4-dihydro-4-(2′-methoxy-3′,5′-diamino-6′-pentadecylphenyl)-2,6-dimethyl-3,5-pyridine dicarboxylate;
diethyl 1,4-dihydro-4-(2′-isopropoxy-3′,5′-diamino-6′-pentadecylphenyl)-2,6-dimethyl-3,5-pyridine dicarboxylate;
dimethyl 1,4-dihydro-4-(2′-isopropoxy-3′,5′-diamino-6′-pentadecylphenyl)-2,6-dimethyl-3,5-pyridine dicarboxylate;
diisopropyl 1,4-dihydro-4-(2′-isopropoxy-3′,5′-diamino-6′-pentadecylphenyl)-2,6-dimethyl-3,5-pyridine dicarboxylate;
diethyl 1,4-dihydro-4-(2′-ethoxy-6′-pentadecylphenyl)-2-methyl-6-(5″-methyl-2-mercapto-1′H-benzimidazolyl)methyl-3,5-pyridine dicarboxylate;
dimethyl 1,4-dihydro-4-(2′-ethoxy-6′-pentadecylphenyl)-2-methyl-6-(5″-methyl-2-mercapto-1′H-benzimidazolyl)methyl-3,5-pyridine dicarboxylate;
diisopropyl 1,4-dihydro-4-(2′-ethoxy-6′-pentadecylphenyl)-2-methyl-6-(5″-methyl-2-mercapto-1′H-benzimidazolyl)methyl-3,5-pyridine dicarboxylate;
diethyl 1,4-dihydro-4-(2′-methoxy-6′-pentadecylphenyl)-2-methyl-6-(5″-methyl-2-mercapto-1′H-benzimidazolyl)methyl-3,5-pyridine dicarboxylate;
dimethyl 1,4-dihydro-4-(2′-methoxy-6′-pentadecylphenyl)-2-methyl-6-(5″-methyl-2-mercapto-1′H-benzimidazolyl)methyl-3,5-pyridine dicarboxylate;
diisopropyl 1,4-dihydro-4-(2′-methoxy-6′-pentadecylphenyl)-2-methyl-6-(5″-methyl-2-mercapto-1′H-benzimidazolyl)methyl-3,5-pyridine dicarboxylate;
diethyl 1,4-dihydro-4-(2′-isopropoxy-6′-pentadecylphenyl)-2-methyl-6-(5″-methyl-2-mercapto-1′H-benzimidazolyl)methyl-3,5-pyridine dicarboxylate;
dimethyl 1,4-dihydro-4-(2′-isopropoxy-6′-pentadecylphenyl)-2-methyl-6-methyl(5′-methyl-2-mercapto-1′H-benzimidazolyl)methyl-3,5-pyridine dicarboxylate; and
diisopropyl 1,4-dihydro-4-(2′-isopropoxy-6′-pentadecylphenyl)-2-methyl-6-methyl (5′-methyl-2-mercapto-1′H-benzimidazolyl)methyl-3,5-pyridine dicarboxylate.

Some of the compounds of the present invention are listed in Table I, below, and are designated DHP-1 through DHP-655.

TABLE I

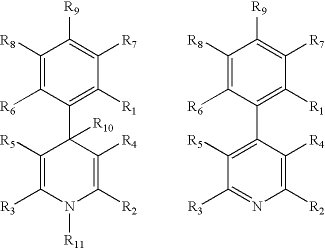

| Comp'd | | | | | | | | | |
|---|---|---|---|---|---|---|---|---|---|
| Number | R₁ | R₂ | R₃ | R₄ | R₅ | R₆ | R₇ | R₈ | R₉ |
| DHP-1 | C₁₅H₃₁ | CH₃ | CH₃ | C(O)OCH₂CH₃ | C(O)OCH₃ | OCH₃ | H | H | H |
| DHP-2 | C₁₅H₃₁ | CH₃ | CH₃ | C(O)OCH₂CH₃ | C(O)OCH₂CH₃ | OCH₃ | H | H | H |
| DHP-3 | C₁₅H₃₁ | CH₃ | CH₃ | C(O)OCH₂CH₃ | C(O)OCH₃ | OCH₂CH₃ | H | H | H |
| DHP-4 | C₁₅H₃₁ | CH₃ | CH₃ | C(O)OCH₂CH₃ | C(O)OCH₂CH₃ | OCH₂CH₃ | H | H | H |
| DHP-5 | C₁₅H₃₁ | CH₃ | CH₃ | C(O)OCH₃ | C(O)OCH₃ | OCH(CH₃)₂ | H | H | H |
| DHP-6 | C₁₅H₃₁ | CH₃ | CH₃ | C(O)OCH₂CH₃ | C(O)OCH₃ | OCH(CH₃)₂ | H | H | H |
| DHP-7 | C₁₅H₃₁ | CH₃ | CH₃ | C(O)OCH₂CH₃ | C(O)OCH₂CH₃ | OCH(CH₃)₂ | H | H | H |
| DHP-8 | C₁₅H₃₁ | CH₃ | CH₃ | C(O)OCH(CH₃)₂ | C(O)OCH₃ | OCH₃ | H | H | H |
| DHP-9 | C₁₅H₃₁ | CH₃ | CH₃ | C(O)OCH(CH₃)₂ | C(O)OCH₂CH₃ | OCH₃ | H | H | H |
| DHP-10 | C₁₅H₃₁ | CH₃ | CH₃ | C(O)OCH(CH₃)₂ | C(O)OCH(CH₃)₂ | OCH₃ | H | H | H |
| DHP-11 | C₁₅H₃₁ | CH₃ | CH₃ | C(O)OCH(CH₃)₂ | C(O)OCH₃ | OCH₂CH₃ | H | H | H |
| DHP-12 | C₁₅H₃₁ | CH₃ | CH₃ | C(O)OCH(CH₃)₂ | C(O)OCH₂CH₃ | OCH₂CH₃ | H | H | H |
| DHP-13 | C₁₅H₃₁ | CH₃ | CH₃ | C(O)OCH(CH₃)₂ | C(O)OCH(CH₃)₂ | OCH₂CH₃ | H | H | H |
| DHP-14 | C₁₅H₃₁ | CH₃ | CH₃ | C(O)OCH(CH₃)₂ | C(O)OCH₃ | OCH(CH₃)₂ | H | H | H |
| DHP-15 | C₁₅H₃₁ | CH₃ | CH₃ | C(O)OCH(CH₃)₂ | C(O)OCH₂CH₃ | OCH(CH₃)₂ | H | H | H |
| DHP-16 | C₁₅H₃₁ | CH₃ | CH₃ | C(O)OCH(CH₃)₂ | C(O)OCH(CH₃)₂ | OCH(CH₃)₂ | H | H | H |
| DHP-17 | C₁₅H₃₁ | CH₃ | CH₃ | C(O)OCH₂CH₃ | C(O)OCH₂CH₃ | OCH₃ | H | H | H |
| DHP-18 | C₁₅H₃₁ | CH₃ | CH₃ | C(O)OCH₂CH₃ | C(O)OCH₂CH₃ | OCH₂CH₃ | H | H | H |
| DHP-19 | C₁₅H₃₁ | CH₃ | CH₃ | C(O)OCH₃ | C(O)OCH₃ | OCH₂CH₃ | H | H | H |
| DHP-20 | C₁₅H₃₁ | CH₃ | CH₃ | C(O)OCH₃ | C(O)OCH₃ | OCH₃ | H | H | H |
| DHP-21 | C₁₅H₃₁ | CH₃ | A* | C(O)OCH₂CH₃ | C(O)OCH₂CH₃ | OCH₃ | H | H | H |
| DHP-22 | C₁₅H₃₁ | CH₃ | A | C(O)OCH₂CH₃ | C(O)OCH₃ | OCH₃ | H | H | H |
| DHP-23 | C₁₅H₃₁ | CH₃ | A | C(O)OCH₂CH₃ | C(O)OCH(CH₃)₂ | OCH₃ | H | H | H |
| DHP-24 | C₁₅H₃₁ | CH₃ | A | C(O)OCH₂CH₃ | C(O)OCH₂CH₃ | OCH(CH₃)₂ | H | H | H |
| DHP-25 | C₁₅H₃₁ | CH₃ | A | C(O)OCH₂CH₃ | C(O)OCH₃ | OCH(CH₃)₂ | H | H | H |
| DHP-26 | C₁₅H₃₁ | CH₃ | A | C(O)OCH₂CH₃ | C(O)OCH(CH₃)₂ | OCH(CH₃)₂ | H | H | H |
| DHP-27 | C₁₅H₃₁ | CH₃ | A | C(O)OCH₂CH₃ | C(O)OCH₂CH₃ | OCH₂CH₃ | H | H | H |
| DHP-28 | C₁₅H₃₁ | CH₃ | A | C(O)OCH₂CH₃ | C(O)OCH₃ | OCH₂CH₃ | H | H | H |
| DHP-29 | C₁₅H₃₁ | CH₃ | A | C(O)OCH₂CH₃ | C(O)OCH(CH₃)₂ | OCH₂CH₃ | H | H | H |
| DHP-30 | C₁₅H₃₁ | CH₃ | A | C(O)OCH₃ | C(O)OCH₃ | OCH₃ | H | H | H |
| DHP-31 | C₁₅H₃₁ | CH₃ | A | C(O)OCH₃ | C(O)OCH₂CH₃ | OCH₃ | H | H | H |
| DHP-32 | C₁₅H₃₁ | CH₃ | A | C(O)OCH₃ | C(O)OCH(CH₃)₂ | OCH₃ | H | H | H |
| DHP-33 | C₁₅H₃₁ | CH₃ | A | C(O)OCH₃ | C(O)OCH₃ | OCH(CH₃)₂ | H | H | H |
| DHP-34 | C₁₅H₃₁ | CH₃ | A | C(O)OCH₃ | C(O)OCH₂CH₃ | OCH(CH₃)₂ | H | H | H |
| DHP-35 | C₁₅H₃₁ | CH₃ | A | C(O)OCH₃ | C(O)OCH(CH₃)₂ | OCH(CH₃)₂ | H | H | H |
| DHP-36 | C₁₅H₃₁ | CH₃ | A | C(O)OCH₃ | C(O)OCH₃ | OCH₂CH₃ | H | H | H |
| DHP-37 | C₁₅H₃₁ | CH₃ | A | C(O)OCH₃ | C(O)OCH₂CH₃ | OCH₂CH₃ | H | H | H |
| DHP-38 | C₁₅H₃₁ | CH₃ | A | C(O)OCH₃ | C(O)OCH(CH₃)₂ | OCH₂CH₃ | H | H | H |
| DHP-39 | C₁₅H₃₁ | CH₃ | A | C(O)OCH(CH₃)₂ | C(O)OCH(CH₃)₂ | OCH₃ | H | H | H |
| DHP-40 | C₁₅H₃₁ | CH₃ | A | C(O)OCH(CH₃)₂ | C(O)OCH₃ | OCH₃ | H | H | H |
| DHP-41 | C₁₅H₃₁ | CH₃ | A | C(O)OCH(CH₃)₂ | C(O)OCH₂CH₃ | OCH₃ | H | H | H |
| DHP-42 | C₁₅H₃₁ | CH₃ | A | C(O)OCH(CH₃)₂ | C(O)OCH(CH₃)₂ | OCH(CH₃)₂ | H | H | H |
| DHP-43 | C₁₅H₃₁ | CH₃ | A | C(O)OCH(CH₃)₂ | C(O)OCH₃ | OCH(CH₃)₂ | H | H | H |
| DHP-44 | C₁₅H₃₁ | CH₃ | A | C(O)OCH(CH₃)₂ | C(O)OCH₂CH₃ | OCH(CH₃)₂ | H | H | H |
| DHP-45 | C₁₅H₃₁ | CH₃ | A | C(O)OCH(CH₃)₂ | C(O)OCH(CH₃)₂ | OCH₂CH₃ | H | H | H |
| DHP-46 | C₁₅H₃₁ | CH₃ | A | C(O)OCH(CH₃)₂ | C(O)OCH₃ | OCH₂CH₃ | H | H | H |
| DHP-47 | C₁₅H₃₁ | CH₃ | A | C(O)OCH(CH₃)₂ | C(O)OCH₂CH₃ | OCH₂CH₃ | H | H | H |
| DHP-48 | C₁₅H₃₁ | CH₃ | A | C(O)OH | C(O)OH | OCH₃ | H | H | H |
| DHP-49 | C₁₅H₃₁ | CH₃ | A | C(O)OH | C(O)OCH₃ | OCH₃ | H | H | H |
| DHP-50 | C₁₅H₃₁ | CH₃ | A | C(O)OH | C(O)OCH₂CH₃ | OCH₃ | H | H | H |
| DHP-51 | C₁₅H₃₁ | CH₃ | A | C(O)OH | C(O)OCH(CH₃)₂ | OCH₃ | H | H | H |
| DHP-52 | C₁₅H₃₁ | CH₃ | A | C(O)OH | C(O)OH | OCH(CH₃)₂ | H | H | H |
| DHP-53 | C₁₅H₃₁ | CH₃ | A | C(O)OH | C(O)OCH₃ | OCH(CH₃)₂ | H | H | H |
| DHP-54 | C₁₅H₃₁ | CH₃ | A | C(O)OH | C(O)OCH₂CH₃ | OCH(CH₃)₂ | H | H | H |
| DHP-55 | C₁₅H₃₁ | CH₃ | A | C(O)OH | C(O)OCH(CH₃)₂ | OCH(CH₃)₂ | H | H | H |
| DHP-56 | C₁₅H₃₁ | CH₃ | A | C(O)OH | C(O)OH | OCH₂CH₃ | H | H | H |
| DHP-57 | C₁₅H₃₁ | CH₃ | A | C(O)OH | C(O)OCH₃ | OCH₂CH₃ | H | H | H |
| DHP-58 | C₁₅H₃₁ | CH₃ | A | C(O)OH | C(O)OCH₂CH₃ | OCH₂CH₃ | H | H | H |
| DHP-59 | C₁₅H₃₁ | CH₃ | A | C(O)OH | C(O)OCH(CH₃)₂ | OCH₂CH₃ | H | H | H |
| DHP-60 | C₁₅H₃₁ | CH₃ | A | C(O)OCH₃ | C(O)OH | OCH₃ | H | H | H |
| DHP-61 | C₁₅H₃₁ | CH₃ | A | C(O)OCH₂CH₃ | C(O)OH | OCH₃ | H | H | H |
| DHP-62 | C₁₅H₃₁ | CH₃ | A | C(O)OCH(CH₃)₂ | C(O)OH | OCH₃ | H | H | H |
| DHP-63 | C₁₅H₃₁ | CH₃ | A | C(O)OCH₃ | C(O)OH | OCH(CH₃)₂ | H | H | H |
| DHP-64 | C₁₅H₃₁ | CH₃ | A | C(O)OCH₂CH₃ | C(O)OH | OCH(CH₃)₂ | H | H | H |
| DHP-65 | C₁₅H₃₁ | CH₃ | A | C(O)OCH(CH₃)₂ | C(O)OH | OCH(CH₃)₂ | H | H | H |
| DHP-66 | C₁₅H₃₁ | CH₃ | A | C(O)OCH₃ | C(O)OH | OCH₂CH₃ | H | H | H |
| DHP-67 | C₁₅H₃₁ | CH₃ | A | C(O)OCH₂CH₃ | C(O)OH | OCH₂CH₃ | H | H | H |
| DHP-68 | C₁₅H₃₁ | CH₃ | A | C(O)OCH(CH₃)₂ | C(O)OH | OCH₂CH₃ | H | H | H |
| DHP-69 | C₁₅H₃₁ | CH₃ | CH₃ | C(O)OCH₂CH₂OCH₃ | C(O)OCH₂CH₃ | OCH₂CH₃ | H | H | H |
| DHP-70 | C₁₅H₃₁ | CH₃ | CH₃ | C(O)OCH₂CH₂OCH₃ | C(O)OCH₃ | OCH₂CH₃ | H | H | H |
| DHP-71 | C₁₅H₃₁ | CH₃ | CH₃ | C(O)OCH₂CH₂OCH₃ | C(O)OCH(CH₃)₂ | OCH₂CH₃ | H | H | H |
| DHP-72 | C₁₅H₃₁ | CH₃ | CH₃ | C(O)OCH₂CH₂OCH₃ | C(O)OCH₂CH₃ | OCH(CH₃)₂ | H | H | H |
| DHP-73 | C₁₅H₃₁ | CH₃ | CH₃ | C(O)OCH₂CH₂OCH₃ | C(O)OCH₃ | OCH(CH₃)₂ | H | H | H |
| DHP-74 | C₁₅H₃₁ | CH₃ | CH₃ | C(O)OCH₂CH₂OCH₃ | C(O)OCH(CH₃)₂ | OCH(CH₃)₂ | H | H | H |
| DHP-75 | C₁₅H₃₁ | CH₃ | CH₃ | C(O)OCH₂CH₂OCH₃ | C(O)OCH₂CH₃ | OCH₃ | H | H | H |
| DHP-76 | C₁₅H₃₁ | CH₃ | CH₃ | C(O)OCH₂CH₂OCH₃ | C(O)OCH₃ | OCH₃ | H | H | H |
| DHP-77 | C₁₅H₃₁ | CH₃ | CH₃ | C(O)OCH₂CH₂OCH₃ | C(O)OCH(CH₃)₂ | OCH₃ | H | H | H |
| DHP-78 | C₁₅H₃₁ | B** | CH₃ | C(O)OCH₂CH₃ | C(O)OCH₂CH₃ | OCH₃ | H | H | H |
| DHP-79 | C₁₅H₃₁ | B | CH₃ | C(O)OCH₂CH₃ | C(O)OCH₃ | OCH₃ | H | H | H |
| DHP-80 | C₁₅H₃₁ | B | CH₃ | C(O)OCH₂CH₃ | C(O)OCH(CH₃)₂ | OCH₃ | H | H | H |
| DHP-81 | C₁₅H₃₁ | B | CH₃ | C(O)OCH₂CH₃ | C(O)OCH₂CH₃ | OCH(CH₃)₂ | H | H | H |
| DHP-82 | C₁₅H₃₁ | B | CH₃ | C(O)OCH₂CH₃ | C(O)OCH₃ | OCH(CH₃)₂ | H | H | H |
| DHP-83 | C₁₅H₃₁ | B | CH₃ | C(O)OCH₂CH₃ | C(O)OCH(CH₃)₂ | OCH(CH₃)₂ | H | H | H |
| DHP-84 | C₁₅H₃₁ | B | CH₃ | C(O)OCH₂CH₃ | C(O)OCH₂CH₃ | OCH₂CH₃ | H | H | H |
| DHP-85 | C₁₅H₃₁ | B | CH₃ | C(O)OCH₂CH₃ | C(O)OCH₃ | OCH₂CH₃ | H | H | H |
| DHP-86 | C₁₅H₃₁ | B | CH₃ | C(O)OCH₂CH₃ | C(O)OCH(CH₃)₂ | OCH₂CH₃ | H | H | H |
| DHP-87 | C₁₅H₃₁ | B | CH₃ | C(O)OCH₃ | C(O)OCH₃ | OCH₃ | H | H | H |
| DHP-88 | C₁₅H₃₁ | B | CH₃ | C(O)OCH₃ | C(O)OCH₂CH₃ | OCH₃ | H | H | H |
| DHP-89 | C₁₅H₃₁ | B | CH₃ | C(O)OCH₃ | C(O)OCH(CH₃)₂ | OCH₃ | H | H | H |
| DHP-90 | C₁₅H₃₁ | B | CH₃ | C(O)OCH₃ | C(O)OCH₃ | OCH(CH₃)₂ | H | H | H |
| DHP-91 | C₁₅H₃₁ | B | CH₃ | C(O)OCH₃ | C(O)OCH₂CH₃ | OCH(CH₃)₂ | H | H | H |
| DHP-92 | C₁₅H₃₁ | B | CH₃ | C(O)OCH₃ | C(O)OCH(CH₃)₂ | OCH(CH₃)₂ | H | H | H |
| DHP-93 | C₁₅H₃₁ | B | CH₃ | C(O)OCH₃ | C(O)OCH₃ | OCH₂CH₃ | H | H | H |
| DHP-94 | C₁₅H₃₁ | B | CH₃ | C(O)OCH₃ | C(O)OCH₂CH₃ | OCH₂CH₃ | H | H | H |
| DHP-95 | C₁₅H₃₁ | B | CH₃ | C(O)OCH₃ | C(O)OCH(CH₃)₂ | OCH₂CH₃ | H | H | H |
| DHP-96 | C₁₅H₃₁ | B | CH₃ | C(O)OCH(CH₃)₂ | C(O)OCH(CH₃)₂ | OCH₃ | H | H | H |
| DHP-97 | C₁₅H₃₁ | B | CH₃ | C(O)OCH(CH₃)₂ | C(O)OCH₃ | OCH₃ | H | H | H |
| DHP-98 | C₁₅H₃₁ | B | CH₃ | C(O)OCH(CH₃)₂ | C(O)OCH₂CH₃ | OCH₃ | H | H | H |
| DHP-99 | C₁₅H₃₁ | B | CH₃ | C(O)OCH(CH₃)₂ | C(O)OCH(CH₃)₂ | OCH(CH₃)₂ | H | H | H |
| DHP-100 | C₁₅H₃₁ | B | CH₃ | C(O)OCH(CH₃)₂ | C(O)OCH₃ | OCH(CH₃)₂ | H | H | H |
| DHP-101 | C₁₅H₃₁ | B | CH₃ | C(O)OCH(CH₃)₂ | C(O)OCH₂CH₃ | OCH(CH₃)₂ | H | H | H |
| DHP-102 | C₁₅H₃₁ | B | CH₃ | C(O)OCH(CH₃)₂ | C(O)OCH(CH₃)₂ | OCH₂CH₃ | H | H | H |
| DHP-103 | C₁₅H₃₁ | B | CH₃ | C(O)OCH(CH₃)₂ | C(O)OCH₃ | OCH₂CH₃ | H | H | H |
| DHP-104 | C₁₅H₃₁ | B | CH₃ | C(O)OCH(CH₃)₂ | C(O)OCH₂CH₃ | OCH₂CH₃ | H | H | H |
| DHP-105 | C₁₅H₃₁ | B | CH₃ | C(O)OH | C(O)OH | OCH₃ | H | H | H |
| DHP-106 | C₁₅H₃₁ | B | CH₃ | C(O)OH | C(O)OCH₃ | OCH₃ | H | H | H |
| DHP-107 | C₁₅H₃₁ | B | CH₃ | C(O)OH | C(O)OCH₂CH₃ | OCH₃ | H | H | H |
| DHP-108 | C₁₅H₃₁ | B | CH₃ | C(O)OH | C(O)OCH(CH₃)₂ | OCH₃ | H | H | H |
| DHP-109 | C₁₅H₃₁ | B | CH₃ | C(O)OH | C(O)OH | OCH(CH₃)₂ | H | H | H |
| DHP-110 | C₁₅H₃₁ | B | CH₃ | C(O)OH | C(O)OCH₃ | OCH(CH₃)₂ | H | H | H |
| DHP-111 | C₁₅H₃₁ | B | CH₃ | C(O)OH | C(O)OCH₂CH₃ | OCH(CH₃)₂ | H | H | H |
| DHP-112 | C₁₅H₃₁ | B | CH₃ | C(O)OH | C(O)OCH(CH₃)₂ | OCH(CH₃)₂ | H | H | H |
| DHP-113 | C₁₅H₃₁ | B | CH₃ | C(O)OH | C(O)OH | OCH₂CH₃ | H | H | H |
| DHP-114 | C₁₅H₃₁ | B | CH₃ | C(O)OH | C(O)OCH₃ | OCH₂CH₃ | H | H | H |
| DHP-115 | C₁₅H₃₁ | B | CH₃ | C(O)OH | C(O)OCH₂CH₃ | OCH₂CH₃ | H | H | H |
| DHP-116 | C₁₅H₃₁ | B | CH₃ | C(O)OH | C(O)OCH(CH₃)₂ | OCH₂CH₃ | H | H | H |
| DHP-117 | C₁₅H₃₁ | B | CH₃ | C(O)OCH₃ | C(O)OH | OCH₃ | H | H | H |
| DHP-118 | C₁₅H₃₁ | B | CH₃ | C(O)OCH₂CH₃ | C(O)OH | OCH₃ | H | H | H |
| DHP-119 | C₁₅H₃₁ | B | CH₃ | C(O)OCH(CH₃)₂ | C(O)OH | OCH₃ | H | H | H |
| DHP-120 | C₁₅H₃₁ | B | CH₃ | C(O)OCH₃ | C(O)OH | OCH(CH₃)₂ | H | H | H |
| DHP-121 | C₁₅H₃₁ | B | CH₃ | C(O)OCH₂CH₃ | C(O)OH | OCH(CH₃)₂ | H | H | H |
| DHP-122 | C₁₅H₃₁ | B | CH₃ | C(O)OCH(CH₃)₂ | C(O)OH | OCH(CH₃)₂ | H | H | H |
| DHP-123 | C₁₅H₃₁ | B | CH₃ | C(O)OCH₃ | C(O)OH | OCH₂CH₃ | H | H | H |
| DHP-124 | C₁₅H₃₁ | B | CH₃ | C(O)OCH₂CH₃ | C(O)OH | OCH₂CH₃ | H | H | H |
| DHP-125 | C₁₅H₃₁ | B | CH₃ | C(O)OCH(CH₃)₂ | C(O)OH | OCH₂CH₃ | H | H | H |
| DHP-126 | C₁₅H₃₁ | CH₃ | CH₃ | C(O)OCH₂CH₃ | C(O)OCH₂CH₃ | OCH₃ | NO₂ | NO₂ | H |
| DHP-127 | C₁₅H₃₁ | CH₃ | CH₃ | C(O)OCH₂CH₃ | C(O)OCH₃ | OCH₃ | NO₂ | NO₂ | H |
| DHP-128 | C₁₅H₃₁ | CH₃ | CH₃ | C(O)OCH₂CH₃ | C(O)OCH(CH₃)₂ | OCH₃ | NO₂ | NO₂ | H |
| DHP-129 | C₁₅H₃₁ | CH₃ | CH₃ | C(O)OCH₂CH₃ | C(O)OCH₂CH₃ | OCH(CH₃)₂ | NO₂ | NO₂ | H |
| DHP-130 | C₁₅H₃₁ | CH₃ | CH₃ | C(O)OCH₂CH₃ | C(O)OCH₃ | OCH(CH₃)₂ | NO₂ | NO₂ | H |
| DHP-131 | C₁₅H₃₁ | CH₃ | CH₃ | C(O)OCH₂CH₃ | C(O)OCH(CH₃)₂ | OCH(CH₃)₂ | NO₂ | NO₂ | H |
| DHP-132 | C₁₅H₃₁ | CH₃ | CH₃ | C(O)OCH₂CH₃ | C(O)OCH₂CH₃ | OCH₂CH₃ | NO₂ | NO₂ | H |
| DHP-133 | C₁₅H₃₁ | CH₃ | CH₃ | C(O)OCH₂CH₃ | C(O)OCH₃ | OCH₂CH₃ | NO₂ | NO₂ | H |
| DHP-134 | C₁₅H₃₁ | CH₃ | CH₃ | C(O)OCH₂CH₃ | C(O)OCH(CH₃)₂ | OCH₂CH₃ | NO₂ | NO₂ | H |
| DHP-135 | C₁₅H₃₁ | CH₃ | CH₃ | C(O)OCH₃ | C(O)OCH₃ | OCH₃ | NO₂ | NO₂ | H |
| DHP-136 | C₁₅H₃₁ | CH₃ | CH₃ | C(O)OCH₃ | C(O)OCH₂CH₃ | OCH₃ | NO₂ | NO₂ | H |
| DHP-137 | C₁₅H₃₁ | CH₃ | CH₃ | C(O)OCH₃ | C(O)OCH(CH₃)₂ | OCH₃ | NO₂ | NO₂ | H |
| DHP-138 | C₁₅H₃₁ | CH₃ | CH₃ | C(O)OCH₃ | C(O)OCH₃ | OCH(CH₃)₂ | NO₂ | NO₂ | H |
| DHP-139 | C₁₅H₃₁ | CH₃ | CH₃ | C(O)OCH₃ | C(O)OCH₂CH₃ | OCH(CH₃)₂ | NO₂ | NO₂ | H |
| DHP-140 | C₁₅H₃₁ | CH₃ | CH₃ | C(O)OCH₃ | C(O)OCH(CH₃)₂ | OCH(CH₃)₂ | NO₂ | NO₂ | H |
| DHP-141 | C₁₅H₃₁ | CH₃ | CH₃ | C(O)OCH₃ | C(O)OCH₃ | OCH₂CH₃ | NO₂ | NO₂ | H |
| DHP-142 | C₁₅H₃₁ | CH₃ | CH₃ | C(O)OCH₃ | C(O)OCH₂CH₃ | OCH₂CH₃ | NO₂ | NO₂ | H |
| DHP-143 | C₁₅H₃₁ | CH₃ | CH₃ | C(O)OCH₃ | C(O)OCH(CH₃)₂ | OCH₂CH₃ | NO₂ | NO₂ | H |
| DHP-144 | C₁₅H₃₁ | CH₃ | CH₃ | C(O)OCH(CH₃)₂ | C(O)OCH(CH₃)₂ | OCH₃ | NO₂ | NO₂ | H |
| DHP-145 | C₁₅H₃₁ | CH₃ | CH₃ | C(O)OCH(CH₃)₂ | C(O)OCH₃ | OCH₃ | NO₂ | NO₂ | H |
| DHP-146 | C₁₅H₃₁ | CH₃ | CH₃ | C(O)OCH(CH₃)₂ | C(O)OCH₂CH₃ | OCH₃ | NO₂ | NO₂ | H |
| DHP-147 | C₁₅H₃₁ | CH₃ | CH₃ | C(O)OCH(CH₃)₂ | C(O)OCH(CH₃)₂ | OCH(CH₃)₂ | NO₂ | NO₂ | H |
| DHP-148 | C₁₅H₃₁ | CH₃ | CH₃ | C(O)OCH(CH₃)₂ | C(O)OCH₃ | OCH(CH₃)₂ | NO₂ | NO₂ | H |
| DHP-149 | C₁₅H₃₁ | CH₃ | CH₃ | C(O)OCH(CH₃)₂ | C(O)OCH₂CH₃ | OCH(CH₃)₂ | NO₂ | NO₂ | H |
| DHP-150 | C₁₅H₃₁ | CH₃ | CH₃ | C(O)OCH(CH₃)₂ | C(O)OCH(CH₃)₂ | OCH₂CH₃ | NO₂ | NO₂ | H |
| DHP-151 | C₁₅H₃₁ | CH₃ | CH₃ | C(O)OCH(CH₃)₂ | C(O)OCH₃ | OCH₂CH₃ | NO₂ | NO₂ | H |
| DHP-152 | C₁₅H₃₁ | CH₃ | CH₃ | C(O)OCH(CH₃)₂ | C(O)OCH₂CH₃ | OCH₂CH₃ | NO₂ | NO₂ | H |
| DHP-153 | C₁₅H₃₁ | CH₃ | CH₃ | C(O)OH | C(O)OH | OCH₃ | NO₂ | NO₂ | H |
| DHP-154 | C₁₅H₃₁ | CH₃ | CH₃ | C(O)OH | C(O)OCH₃ | OCH₃ | NO₂ | NO₂ | H |
| DHP-155 | C₁₅H₃₁ | CH₃ | CH₃ | C(O)OH | C(O)OCH₂CH₃ | OCH₃ | NO₂ | NO₂ | H |
| DHP-156 | C₁₅H₃₁ | CH₃ | CH₃ | C(O)OH | C(O)OCH(CH₃)₂ | OCH₃ | NO₂ | NO₂ | H |
| DHP-157 | C₁₅H₃₁ | CH₃ | CH₃ | C(O)OH | C(O)OH | OCH(CH₃)₂ | NO₂ | NO₂ | H |
| DHP-158 | C₁₅H₃₁ | CH₃ | CH₃ | C(O)OH | C(O)OCH₃ | OCH(CH₃)₂ | NO₂ | NO₂ | H |
| DHP-159 | C₁₅H₃₁ | CH₃ | CH₃ | C(O)OH | C(O)OCH₂CH₃ | OCH(CH₃)₂ | NO₂ | NO₂ | H |
| DHP-160 | C₁₅H₃₁ | CH₃ | CH₃ | C(O)OH | C(O)OCH(CH₃)₂ | OCH(CH₃)₂ | NO₂ | NO₂ | H |
| DHP-161 | C₁₅H₃₁ | CH₃ | CH₃ | C(O)OH | C(O)OH | OCH₂CH₃ | NO₂ | NO₂ | H |
| DHP-162 | C₁₅H₃₁ | CH₃ | CH₃ | C(O)OH | C(O)OCH₃ | OCH₂CH₃ | NO₂ | NO₂ | H |
| DHP-163 | C₁₅H₃₁ | CH₃ | CH₃ | C(O)OH | C(O)OCH₂CH₃ | OCH₂CH₃ | NO₂ | NO₂ | H |
| DHP-164 | C₁₅H₃₁ | CH₃ | CH₃ | C(O)OH | C(O)OCH(CH₃)₂ | OCH₂CH₃ | NO₂ | NO₂ | H |
| DHP-165 | C₁₅H₃₁ | CH₃ | CH₃ | C(O)OCH₃ | C(O)OH | OCH₃ | NO₂ | NO₂ | H |
| DHP-166 | C₁₅H₃₁ | CH₃ | CH₃ | C(O)OCH₂CH₃ | C(O)OH | OCH₃ | NO₂ | NO₂ | H |
| DHP-167 | C₁₅H₃₁ | CH₃ | CH₃ | C(O)OCH(CH₃)₂ | C(O)OH | OCH₃ | NO₂ | NO₂ | H |
| DHP-168 | C₁₅H₃₁ | CH₃ | CH₃ | C(O)OCH₃ | C(O)OH | OCH(CH₃)₂ | NO₂ | NO₂ | H |
| DHP-169 | C₁₅H₃₁ | CH₃ | CH₃ | C(O)OCH₂CH₃ | C(O)OH | OCH(CH₃)₂ | NO₂ | NO₂ | H |
| DHP-170 | C₁₅H₃₁ | CH₃ | CH₃ | C(O)OCH(CH₃)₂ | C(O)OH | OCH(CH₃)₂ | NO₂ | NO₂ | H |
| DHP-171 | C₁₅H₃₁ | CH₃ | CH₃ | C(O)OCH₃ | C(O)OH | OCH₂CH₃ | NO₂ | NO₂ | H |
| DHP-172 | C₁₅H₃₁ | CH₃ | CH₃ | C(O)OCH₂CH₃ | C(O)OH | OCH₂CH₃ | NO₂ | NO₂ | H |
| DHP-173 | C₁₅H₃₁ | CH₃ | CH₃ | C(O)OCH(CH₃)₂ | C(O)OH | OCH₂CH₃ | NO₂ | NO₂ | H |
| DHP-174 | C₁₅H₃₁ | CH₃ | CH₃ | C(O)OCH₂CH₃ | C(O)OCH₂CH₃ | OCH₃ | NO₂ | H | H |
| DHP-175 | C₁₅H₃₁ | CH₃ | CH₃ | C(O)OCH₂CH₃ | C(O)OCH₃ | OCH₃ | NO₂ | H | H |
| DHP-176 | C₁₅H₃₁ | CH₃ | CH₃ | C(O)OCH₂CH₃ | C(O)OCH(CH₃)₂ | OCH₃ | NO₂ | H | H |
| DHP-177 | C₁₅H₃₁ | CH₃ | CH₃ | C(O)OCH₂CH₃ | C(O)OCH₂CH₃ | OCH(CH₃)₂ | NO₂ | H | H |
| DHP-178 | C₁₅H₃₁ | CH₃ | CH₃ | C(O)OCH₂CH₃ | C(O)OCH₃ | OCH(CH₃)₂ | NO₂ | H | H |
| DHP-179 | C₁₅H₃₁ | CH₃ | CH₃ | C(O)OCH₂CH₃ | C(O)OCH(CH₃)₂ | OCH(CH₃)₂ | NO₂ | H | H |
| DHP-180 | C₁₅H₃₁ | CH₃ | CH₃ | C(O)OCH₂CH₃ | C(O)OCH₂CH₃ | OCH₂CH₃ | NO₂ | H | H |
| DHP-181 | C₁₅H₃₁ | CH₃ | CH₃ | C(O)OCH₂CH₃ | C(O)OCH₃ | OCH₂CH₃ | NO₂ | H | H |
| DHP-182 | C₁₅H₃₁ | CH₃ | CH₃ | C(O)OCH₂CH₃ | C(O)OCH(CH₃)₂ | OCH₂CH₃ | NO₂ | H | H |
| DHP-183 | C₁₅H₃₁ | CH₃ | CH₃ | C(O)OCH₃ | C(O)OCH₃ | OCH₃ | NO₂ | H | H |
| DHP-184 | C₁₅H₃₁ | CH₃ | CH₃ | C(O)OCH₃ | C(O)OCH₂CH₃ | OCH₃ | NO₂ | H | H |
| DHP-185 | C₁₅H₃₁ | CH₃ | CH₃ | C(O)OCH₃ | C(O)OCH(CH₃)₂ | OCH₃ | NO₂ | H | H |
| DHP-186 | C₁₅H₃₁ | CH₃ | CH₃ | C(O)OCH₃ | C(O)OCH₃ | OCH(CH₃)₂ | NO₂ | H | H |
| DHP-187 | C₁₅H₃₁ | CH₃ | CH₃ | C(O)OCH₃ | C(O)OCH₂CH₃ | OCH(CH₃)₂ | NO₂ | H | H |
| DHP-188 | C₁₅H₃₁ | CH₃ | CH₃ | C(O)OCH₃ | C(O)OCH(CH₃)₂ | OCH(CH₃)₂ | NO₂ | H | H |
| DHP-189 | C₁₅H₃₁ | CH₃ | CH₃ | C(O)OCH₃ | C(O)OCH₃ | OCH₂CH₃ | NO₂ | H | H |
| DHP-190 | C₁₅H₃₁ | CH₃ | CH₃ | C(O)OCH₃ | C(O)OCH₂CH₃ | OCH₂CH₃ | NO₂ | H | H |
| DHP-191 | C₁₅H₃₁ | CH₃ | CH₃ | C(O)OCH₃ | C(O)OCH(CH₃)₂ | OCH₂CH₃ | NO₂ | H | H |
| DHP-192 | C₁₅H₃₁ | CH₃ | CH₃ | C(O)OCH(CH₃)₂ | C(O)OCH(CH₃)₂ | OCH₃ | NO₂ | H | H |
| DHP-193 | C₁₅H₃₁ | CH₃ | CH₃ | C(O)OCH(CH₃)₂ | C(O)OCH₃ | OCH₃ | NO₂ | H | H |
| DHP-194 | C₁₅H₃₁ | CH₃ | CH₃ | C(O)OCH(CH₃)₂ | C(O)OCH₂CH₃ | OCH₃ | NO₂ | H | H |
| DHP-195 | C₁₅H₃₁ | CH₃ | CH₃ | C(O)OCH(CH₃)₂ | C(O)OCH(CH₃)₂ | OCH(CH₃)₂ | NO₂ | H | H |
| DHP-196 | C₁₅H₃₁ | CH₃ | CH₃ | C(O)OCH(CH₃)₂ | C(O)OCH₃ | OCH(CH₃)₂ | NO₂ | H | H |
| DHP-197 | C₁₅H₃₁ | CH₃ | CH₃ | C(O)OCH(CH₃)₂ | C(O)OCH₂CH₃ | OCH(CH₃)₂ | NO₂ | H | H |
| DHP-198 | C₁₅H₃₁ | CH₃ | CH₃ | C(O)OCH(CH₃)₂ | C(O)OCH(CH₃)₂ | OCH₂CH₃ | NO₂ | H | H |
| DHP-199 | C₁₅H₃₁ | CH₃ | CH₃ | C(O)OCH(CH₃)₂ | C(O)OCH₃ | OCH₂CH₃ | NO₂ | H | H |
| DHP-200 | C₁₅H₃₁ | CH₃ | CH₃ | C(O)OCH(CH₃)₂ | C(O)OCH₂CH₃ | OCH₂CH₃ | NO₂ | H | H |
| DHP-201 | C₁₅H₃₁ | CH₃ | CH₃ | C(O)OH | C(O)OH | OCH₃ | NO₂ | H | H |
| DHP-202 | C₁₅H₃₁ | CH₃ | CH₃ | C(O)OH | C(O)OCH₃ | OCH₃ | NO₂ | H | H |
| DHP-203 | C₁₅H₃₁ | CH₃ | CH₃ | C(O)OH | C(O)OCH₂CH₃ | OCH₃ | NO₂ | H | H |
| DHP-204 | C₁₅H₃₁ | CH₃ | CH₃ | C(O)OH | C(O)OCH(CH₃)₂ | OCH₃ | NO₂ | H | H |
| DHP-205 | C₁₅H₃₁ | CH₃ | CH₃ | C(O)OH | C(O)OH | OCH(CH₃)₂ | NO₂ | H | H |
| DHP-206 | C₁₅H₃₁ | CH₃ | CH₃ | C(O)OH | C(O)OCH₃ | OCH(CH₃)₂ | NO₂ | H | H |
| DHP-207 | C₁₅H₃₁ | CH₃ | CH₃ | C(O)OH | C(O)OCH₂CH₃ | OCH(CH₃)₂ | NO₂ | H | H |
| DHP-208 | C₁₅H₃₁ | CH₃ | CH₃ | C(O)OH | C(O)OCH(CH₃)₂ | OCH(CH₃)₂ | NO₂ | H | H |
| DHP-209 | C₁₅H₃₁ | CH₃ | CH₃ | C(O)OH | C(O)OH | OCH₂CH₃ | NO₂ | H | H |
| DHP-210 | C₁₅H₃₁ | CH₃ | CH₃ | C(O)OH | C(O)OCH₃ | OCH₂CH₃ | NO₂ | H | H |
| DHP-211 | C₁₅H₃₁ | CH₃ | CH₃ | C(O)OH | C(O)OCH₂CH₃ | OCH₂CH₃ | NO₂ | H | H |
| DHP-212 | C₁₅H₃₁ | CH₃ | CH₃ | C(O)OH | C(O)OCH(CH₃)₂ | OCH₂CH₃ | NO₂ | H | H |
| DHP-213 | C₁₅H₃₁ | CH₃ | CH₃ | C(O)OCH₃ | C(O)OH | OCH₃ | NO₂ | H | H |
| DHP-214 | C₁₅H₃₁ | CH₃ | CH₃ | C(O)OCH₂CH₃ | C(O)OH | OCH₃ | NO₂ | H | H |
| DHP-215 | C₁₅H₃₁ | CH₃ | CH₃ | C(O)OCH(CH₃)₂ | C(O)OH | OCH₃ | NO₂ | H | H |
| DHP-216 | C₁₅H₃₁ | CH₃ | CH₃ | C(O)OCH₃ | C(O)OH | OCH(CH₃)₂ | NO₂ | H | H |
| DHP-217 | C₁₅H₃₁ | CH₃ | CH₃ | C(O)OCH₂CH₃ | C(O)OH | OCH(CH₃)₂ | NO₂ | H | H |
| DHP-218 | C₁₅H₃₁ | CH₃ | CH₃ | C(O)OCH(CH₃)₂ | C(O)OH | OCH(CH₃)₂ | NO₂ | H | H |
| DHP-219 | C₁₅H₃₁ | CH₃ | CH₃ | C(O)OCH₃ | C(O)OH | OCH₂CH₃ | NO₂ | H | H |
| DHP-220 | C₁₅H₃₁ | CH₃ | CH₃ | C(O)OCH₂CH₃ | C(O)OH | OCH₂CH₃ | NO₂ | H | H |
| DHP-221 | C₁₅H₃₁ | CH₃ | CH₃ | C(O)OCH(CH₃)₂ | C(O)OH | OCH₂CH₃ | NO₂ | H | H |
| DHP-222 | C₁₅H₃₁ | CH₃ | CH₃ | C(O)OCH₂CH₃ | C(O)OCH₂CH₃ | OCH₃ | H | NO₂ | H |
| DHP-223 | C₁₅H₃₁ | CH₃ | CH₃ | C(O)OCH₂CH₃ | C(O)OCH₃ | OCH₃ | H | NO₂ | H |
| DHP-224 | C₁₅H₃₁ | CH₃ | CH₃ | C(O)OCH₂CH₃ | C(O)OCH(CH₃)₂ | OCH₃ | H | NO₂ | H |
| DHP-225 | C₁₅H₃₁ | CH₃ | CH₃ | C(O)OCH₂CH₃ | C(O)OCH₂CH₃ | OCH(CH₃)₂ | H | NO₂ | H |
| DHP-226 | C₁₅H₃₁ | CH₃ | CH₃ | C(O)OCH₂CH₃ | C(O)OCH₃ | OCH(CH₃)₂ | H | NO₂ | H |
| DHP-227 | C₁₅H₃₁ | CH₃ | CH₃ | C(O)OCH₂CH₃ | C(O)OCH(CH₃)₂ | OCH(CH₃)₂ | H | NO₂ | H |
| DHP-228 | C₁₅H₃₁ | CH₃ | CH₃ | C(O)OCH₂CH₃ | C(O)OCH₂CH₃ | OCH₂CH₃ | H | NO₂ | H |
| DHP-229 | C₁₅H₃₁ | CH₃ | CH₃ | C(O)OCH₂CH₃ | C(O)OCH₃ | OCH₂CH₃ | H | NO₂ | H |
| DHP-230 | C₁₅H₃₁ | CH₃ | CH₃ | C(O)OCH₂CH₃ | C(O)OCH(CH₃)₂ | OCH₂CH₃ | H | NO₂ | H |
| DHP-231 | C₁₅H₃₁ | CH₃ | CH₃ | C(O)OCH₃ | C(O)OCH₃ | OCH₃ | H | NO₂ | H |
| DHP-232 | C₁₅H₃₁ | CH₃ | CH₃ | C(O)OCH₃ | C(O)OCH₂CH₃ | OCH₃ | H | NO₂ | H |
| DHP-233 | C₁₅H₃₁ | CH₃ | CH₃ | C(O)OCH₃ | C(O)OCH(CH₃)₂ | OCH₃ | H | NO₂ | H |
| DHP-234 | C₁₅H₃₁ | CH₃ | CH₃ | C(O)OCH₃ | C(O)OCH₃ | OCH(CH₃)₂ | H | NO₂ | H |
| DHP-235 | C₁₅H₃₁ | CH₃ | CH₃ | C(O)OCH₃ | C(O)OCH₂CH₃ | OCH(CH₃)₂ | H | NO₂ | H |
| DHP-236 | C₁₅H₃₁ | CH₃ | CH₃ | C(O)OCH₃ | C(O)OCH(CH₃)₂ | OCH(CH₃)₂ | H | NO₂ | H |
| DHP-237 | C₁₅H₃₁ | CH₃ | CH₃ | C(O)OCH₃ | C(O)OCH₃ | OCH₂CH₃ | H | NO₂ | H |
| DHP-238 | C₁₅H₃₁ | CH₃ | CH₃ | C(O)OCH₃ | C(O)OCH₂CH₃ | OCH₂CH₃ | H | NO₂ | H |
| DHP-239 | C₁₅H₃₁ | CH₃ | CH₃ | C(O)OCH₃ | C(O)OCH(CH₃)₂ | OCH₂CH₃ | H | NO₂ | H |
| DHP-240 | C₁₅H₃₁ | CH₃ | CH₃ | C(O)OCH(CH₃)₂ | C(O)OCH(CH₃)₂ | OCH₃ | H | NO₂ | H |
| DHP-241 | C₁₅H₃₁ | CH₃ | CH₃ | C(O)OCH(CH₃)₂ | C(O)OCH₃ | OCH₃ | H | NO₂ | H |
| DHP-242 | C₁₅H₃₁ | CH₃ | CH₃ | C(O)OCH(CH₃)₂ | C(O)OCH₂CH₃ | OCH₃ | H | NO₂ | H |
| DHP-243 | C₁₅H₃₁ | CH₃ | CH₃ | C(O)OCH(CH₃)₂ | C(O)OCH(CH₃)₂ | OCH(CH₃)₂ | H | NO₂ | H |
| DHP-244 | C₁₅H₃₁ | CH₃ | CH₃ | C(O)OCH(CH₃)₂ | C(O)OCH₃ | OCH(CH₃)₂ | H | NO₂ | H |
| DHP-245 | C₁₅H₃₁ | CH₃ | CH₃ | C(O)OCH(CH₃)₂ | C(O)OCH₂CH₃ | OCH(CH₃)₂ | H | NO₂ | H |
| DHP-246 | C₁₅H₃₁ | CH₃ | CH₃ | C(O)OCH(CH₃)₂ | C(O)OCH(CH₃)₂ | OCH₂CH₃ | H | NO₂ | H |
| DHP-247 | C₁₅H₃₁ | CH₃ | CH₃ | C(O)OCH(CH₃)₂ | C(O)OCH₃ | OCH₂CH₃ | H | NO₂ | H |
| DHP-248 | C₁₅H₃₁ | CH₃ | CH₃ | C(O)OCH(CH₃)₂ | C(O)OCH₂CH₃ | OCH₂CH₃ | H | NO₂ | H |
| DHP-249 | C₁₅H₃₁ | CH₃ | CH₃ | C(O)OH | C(O)OH | OCH₃ | H | NO₂ | H |
| DHP-250 | C₁₅H₃₁ | CH₃ | CH₃ | C(O)OH | C(O)OCH₃ | OCH₃ | H | NO₂ | H |
| DHP-251 | C₁₅H₃₁ | CH₃ | CH₃ | C(O)OH | C(O)OCH₂CH₃ | OCH₃ | H | NO₂ | H |
| DHP-252 | C₁₅H₃₁ | CH₃ | CH₃ | C(O)OH | C(O)OCH(CH₃)₂ | OCH₃ | H | NO₂ | H |
| DHP-253 | C₁₅H₃₁ | CH₃ | CH₃ | C(O)OH | C(O)OH | OCH(CH₃)₂ | H | NO₂ | H |
| DHP-254 | C₁₅H₃₁ | CH₃ | CH₃ | C(O)OH | C(O)OCH₃ | OCH(CH₃)₂ | H | NO₂ | H |
| DHP-255 | C₁₅H₃₁ | CH₃ | CH₃ | C(O)OH | C(O)OCH₂CH₃ | OCH(CH₃)₂ | H | NO₂ | H |
| DHP-256 | C₁₅H₃₁ | CH₃ | CH₃ | C(O)OH | C(O)OCH(CH₃)₂ | OCH(CH₃)₂ | H | NO₂ | H |
| DHP-257 | C₁₅H₃₁ | CH₃ | CH₃ | C(O)OH | C(O)OH | OCH₂CH₃ | H | NO₂ | H |
| DHP-258 | C₁₅H₃₁ | CH₃ | CH₃ | C(O)OH | C(O)OCH₃ | OCH₂CH₃ | H | NO₂ | H |
| DHP-259 | C₁₅H₃₁ | CH₃ | CH₃ | C(O)OH | C(O)OCH₂CH₃ | OCH₂CH₃ | H | NO₂ | H |
| DHP-260 | C₁₅H₃₁ | CH₃ | CH₃ | C(O)OH | C(O)OCH(CH₃)₂ | OCH₂CH₃ | H | NO₂ | H |
| DHP-261 | C₁₅H₃₁ | CH₃ | CH₃ | C(O)OCH₃ | C(O)OH | OCH₃ | H | NO₂ | H |
| DHP-262 | C₁₅H₃₁ | CH₃ | CH₃ | C(O)OCH₂CH₃ | C(O)OH | OCH₃ | H | NO₂ | H |
| DHP-263 | C₁₅H₃₁ | CH₃ | CH₃ | C(O)OCH(CH₃)₂ | C(O)OH | OCH₃ | H | NO₂ | H |
| DHP-264 | C₁₅H₃₁ | CH₃ | CH₃ | C(O)OCH₃ | C(O)OH | OCH(CH₃)₂ | H | NO₂ | H |
| DHP-265 | C₁₅H₃₁ | CH₃ | CH₃ | C(O)OCH₂CH₃ | C(O)OH | OCH(CH₃)₂ | H | NO₂ | H |
| DHP-266 | C₁₅H₃₁ | CH₃ | CH₃ | C(O)OCH(CH₃)₂ | C(O)OH | OCH(CH₃)₂ | H | NO₂ | H |
| DHP-267 | C₁₅H₃₁ | CH₃ | CH₃ | C(O)OCH₃ | C(O)OH | OCH₂CH₃ | H | NO₂ | H |
| DHP-268 | C₁₅H₃₁ | CH₃ | CH₃ | C(O)OCH₂CH₃ | C(O)OH | OCH₂CH₃ | H | NO₂ | H |
| DHP-269 | C₁₅H₃₁ | CH₃ | CH₃ | C(O)OCH(CH₃)₂ | C(O)OH | OCH₂CH₃ | H | NO₂ | H |
| DHP-270 | C₁₅H₃₁ | CH₃ | C# | C(O)OCH₂CH₃ | C(O)OCH₂CH₃ | OCH₃ | H | H | H |
| DHP-271 | C₁₅H₃₁ | CH₃ | C | C(O)OCH₂CH₃ | C(O)OCH₃ | OCH₃ | H | H | H |
| DHP-272 | C₁₅H₃₁ | CH₃ | C | C(O)OCH₂CH₃ | C(O)OCH(CH₃)₂ | OCH₃ | H | H | H |
| DHP-273 | C₁₅H₃₁ | CH₃ | C | C(O)OCH₂CH₃ | C(O)OCH₂CH₃ | OCH(CH₃)₂ | H | H | H |
| DHP-274 | C₁₅H₃₁ | CH₃ | C | C(O)OCH₂CH₃ | C(O)OCH₃ | OCH(CH₃)₂ | H | H | H |
| DHP-275 | C₁₅H₃₁ | CH₃ | C | C(O)OCH₂CH₃ | C(O)OCH(CH₃)₂ | OCH(CH₃)₂ | H | H | H |
| DHP-276 | C₁₅H₃₁ | CH₃ | C | C(O)OCH₂CH₃ | C(O)OCH₂CH₃ | OCH₂CH₃ | H | H | H |
| DHP-277 | C₁₅H₃₁ | CH₃ | C | C(O)OCH₂CH₃ | C(O)OCH₃ | OCH₂CH₃ | H | H | H |
| DHP-278 | C₁₅H₃₁ | CH₃ | C | C(O)OCH₂CH₃ | C(O)OCH(CH₃)₂ | OCH₂CH₃ | H | H | H |
| DHP-279 | C₁₅H₃₁ | CH₃ | C | C(O)OCH₃ | C(O)OCH₃ | OCH₃ | H | H | H |
| DHP-280 | C₁₅H₃₁ | CH₃ | C | C(O)OCH₃ | C(O)OCH₂CH₃ | OCH₃ | H | H | H |
| DHP-281 | C₁₅H₃₁ | CH₃ | C | C(O)OCH₃ | C(O)OCH(CH₃)₂ | OCH₃ | H | H | H |
| DHP-282 | C₁₅H₃₁ | CH₃ | C | C(O)OCH₃ | C(O)OCH₃ | OCH(CH₃)₂ | H | H | H |
| DHP-283 | C₁₅H₃₁ | CH₃ | C | C(O)OCH₃ | C(O)OCH₂CH₃ | OCH(CH₃)₂ | H | H | H |
| DHP-284 | C₁₅H₃₁ | CH₃ | C | C(O)OCH₃ | C(O)OCH(CH₃)₂ | OCH(CH₃)₂ | H | H | H |
| DHP-285 | C₁₅H₃₁ | CH₃ | C | C(O)OCH₃ | C(O)OCH₃ | OCH₂CH₃ | H | H | H |
| DHP-286 | C₁₅H₃₁ | CH₃ | C | C(O)OCH₃ | C(O)OCH₂CH₃ | OCH₂CH₃ | H | H | H |
| DHP-287 | C₁₅H₃₁ | CH₃ | C | C(O)OCH₃ | C(O)OCH(CH₃)₂ | OCH₂CH₃ | H | H | H |
| DHP-288 | C₁₅H₃₁ | CH₃ | C | C(O)OCH(CH₃)₂ | C(O)OCH(CH₃)₂ | OCH₃ | H | H | H |
| DHP-289 | C₁₅H₃₁ | CH₃ | C | C(O)OCH(CH₃)₂ | C(O)OCH₃ | OCH₃ | H | H | H |
| DHP-290 | C₁₅H₃₁ | CH₃ | C | C(O)OCH(CH₃)₂ | C(O)OCH₂CH₃ | OCH₃ | H | H | H |
| DHP-291 | C₁₅H₃₁ | CH₃ | C | C(O)OCH(CH₃)₂ | C(O)OCH(CH₃)₂ | OCH(CH₃)₂ | H | H | H |
| DHP-292 | C₁₅H₃₁ | CH₃ | C | C(O)OCH(CH₃)₂ | C(O)OCH₃ | OCH(CH₃)₂ | H | H | H |
| DHP-293 | C₁₅H₃₁ | CH₃ | C | C(O)OCH(CH₃)₂ | C(O)OCH₂CH₃ | OCH(CH₃)₂ | H | H | H |
| DHP-294 | C₁₅H₃₁ | CH₃ | C | C(O)OCH(CH₃)₂ | C(O)OCH(CH₃)₂ | OCH₂CH₃ | H | H | H |
| DHP-295 | C₁₅H₃₁ | CH₃ | C | C(O)OCH(CH₃)₂ | C(O)OCH₃ | OCH₂CH₃ | H | H | H |
| DHP-296 | C₁₅H₃₁ | CH₃ | C | C(O)OCH(CH₃)₂ | C(O)OCH₂CH₃ | OCH₂CH₃ | H | H | H |
| DHP-297 | C₁₅H₃₁ | CH₃ | C | C(O)OH | C(O)OH | OCH₃ | H | H | H |
| DHP-298 | C₁₅H₃₁ | CH₃ | C | C(O)OH | C(O)OCH₃ | OCH₃ | H | H | H |
| DHP-299 | C₁₅H₃₁ | CH₃ | C | C(O)OH | C(O)OCH₂CH₃ | OCH₃ | H | H | H |
| DHP-300 | C₁₅H₃₁ | CH₃ | C | C(O)OH | C(O)OCH(CH₃)₂ | OCH₃ | H | H | H |
| DHP-301 | C₁₅H₃₁ | CH₃ | C | C(O)OH | C(O)OH | OCH(CH₃)₂ | H | H | H |
| DHP-302 | C₁₅H₃₁ | CH₃ | C | C(O)OH | C(O)OCH₃ | OCH(CH₃)₂ | H | H | H |
| DHP-303 | C₁₅H₃₁ | CH₃ | C | C(O)OH | C(O)OCH₂CH₃ | OCH(CH₃)₂ | H | H | H |
| DHP-304 | C₁₅H₃₁ | CH₃ | C | C(O)OH | C(O)OCH(CH₃)₂ | OCH(CH₃)₂ | H | H | H |
| DHP-305 | C₁₅H₃₁ | CH₃ | C | C(O)OH | C(O)OH | OCH₂CH₃ | H | H | H |
| DHP-306 | C₁₅H₃₁ | CH₃ | C | C(O)OH | C(O)OCH₃ | OCH₂CH₃ | H | H | H |
| DHP-307 | C₁₅H₃₁ | CH₃ | C | C(O)OH | C(O)OCH₂CH₃ | OCH₂CH₃ | H | H | H |
| DHP-308 | C₁₅H₃₁ | CH₃ | C | C(O)OH | C(O)OCH(CH₃)₂ | OCH₂CH₃ | H | H | H |
| DHP-309 | C₁₅H₃₁ | CH₃ | C | C(O)OCH₃ | C(O)OH | OCH₃ | H | H | H |
| DHP-310 | C₁₅H₃₁ | CH₃ | C | C(O)OCH₂CH₃ | C(O)OH | OCH₃ | H | H | H |
| DHP-311 | C₁₅H₃₁ | CH₃ | C | C(O)OCH(CH₃)₂ | C(O)OH | OCH₃ | H | H | H |
| DHP-312 | C₁₅H₃₁ | CH₃ | C | C(O)OCH₃ | C(O)OH | OCH(CH₃)₂ | H | H | H |
| DHP-313 | C₁₅H₃₁ | CH₃ | C | C(O)OCH₂CH₃ | C(O)OH | OCH(CH₃)₂ | H | H | H |
| DHP-314 | C₁₅H₃₁ | CH₃ | C | C(O)OCH(CH₃)₂ | C(O)OH | OCH(CH₃)₂ | H | H | H |
| DHP-315 | C₁₅H₃₁ | CH₃ | C | C(O)OCH₃ | C(O)OH | OCH₂CH₃ | H | H | H |
| DHP-316 | C₁₅H₃₁ | CH₃ | C | C(O)OCH₂CH₃ | C(O)OH | OCH₂CH₃ | H | H | H |
| DHP-317 | C₁₅H₃₁ | CH₃ | C | C(O)OCH(CH₃)₂ | C(O)OH | OCH₂CH₃ | H | H | H |
| DHP-318 | C₁₅H₃₁ | CH₃ | CH₃ | C(O)OCH₃ | C(O)OCH₃ | OCH₃ | H | H | H |
| DHP-319 | C₁₅H₃₁ | CH₃ | CH₃ | C(O)OCH₃ | C(O)OCH₂CH₃ | OCH₃ | H | H | H |
| DHP-320 | C₁₅H₃₁ | CH₃ | CH₃ | C(O)OCH₃ | C(O)OCH(CH₃)₂ | OCH₃ | H | H | H |
| DHP-321 | C₁₅H₃₁ | CH₃ | CH₃ | C(O)OCH₂CH₃ | C(O)OCH₃ | OCH(CH₃)₂ | H | H | H |
| DHP-322 | C₁₅H₃₁ | CH₃ | CH₃ | C(O)OCH₂CH₃ | C(O)OCH₂CH₃ | OCH(CH₃)₂ | H | H | H |
| DHP-323 | C₁₅H₃₁ | CH₃ | CH₃ | C(O)OCH₂CH₃ | C(O)OCH(CH₃)₂ | OCH(CH₃)₂ | H | H | H |
| DHP-324 | C₁₅H₃₁ | CH₃ | CH₃ | C(O)OCH(CH₃)₂ | C(O)OCH₃ | OCH₂CH₃ | H | H | H |
| DHP-325 | C₁₅H₃₁ | CH₃ | CH₃ | C(O)OCH(CH₃)₂ | C(O)OCH₂CH₃ | OCH₂CH₃ | H | H | H |
| DHP-326 | C₁₅H₃₁ | CH₃ | CH₃ | C(O)OCH(CH₃)₂ | C(O)OCH(CH₃)₂ | OCH₂CH₃ | H | H | H |
| DHP-327 | C₁₀H₂₁ | CH₃ | CH₃ | C(O)OCH₂CH₃ | C(O)OCH₃ | OCH₃ | H | H | H |
| DHP-328 | C₁₀H₂₁ | CH₃ | CH₃ | C(O)OCH₂CH₃ | C(O)OCH₂CH₃ | OCH₃ | H | H | H |
| DHP-329 | C₁₀H₂₁ | CH₃ | CH₃ | C(O)OCH₂CH₃ | C(O)OCH₃ | OCH₂CH₃ | H | H | H |
| DHP-330 | C₁₀H₂₁ | CH₃ | CH₃ | C(O)OCH₂CH₃ | C(O)OCH₂CH₃ | OCH₂CH₃ | H | H | H |
| DHP-331 | C₁₀H₂₁ | CH₃ | CH₃ | C(O)OCH₃ | C(O)OCH₃ | OCH(CH₃)₂ | H | H | H |
| DHP-332 | C₁₀H₂₁ | CH₃ | CH₃ | C(O)OCH₂CH₃ | C(O)OCH₃ | OCH(CH₃)₂ | H | H | H |
| DHP-333 | C₁₀H₂₁ | CH₃ | CH₃ | C(O)OCH₂CH₃ | C(O)OCH₂CH₃ | OCH(CH₃)₂ | H | H | H |
| DHP-334 | C₁₀H₂₁ | CH₃ | CH₃ | C(O)OCH(CH₃)₂ | C(O)OCH₃ | OCH₃ | H | H | H |
| DHP-335 | C₁₀H₂₁ | CH₃ | CH₃ | C(O)OCH(CH₃)₂ | C(O)OCH₂CH₃ | OCH₃ | H | H | H |
| DHP-336 | C₁₀H₂₁ | CH₃ | CH₃ | C(O)OCH(CH₃)₂ | C(O)OCH(CH₃)₂ | OCH₃ | H | H | H |
| DHP-337 | C₁₀H₂₁ | CH₃ | CH₃ | C(O)OCH(CH₃)₂ | C(O)OCH₃ | OCH₂CH₃ | H | H | H |
| DHP-338 | C₁₀H₂₁ | CH₃ | CH₃ | C(O)OCH(CH₃)₂ | C(O)OCH₂CH₃ | OCH₂CH₃ | H | H | H |
| DHP-339 | C₁₀H₂₁ | CH₃ | CH₃ | C(O)OCH(CH₃)₂ | C(O)OCH(CH₃)₂ | OCH₂CH₃ | H | H | H |
| DHP-340 | C₁₀H₂₁ | CH₃ | CH₃ | C(O)OCH(CH₃)₂ | C(O)OCH₃ | OCH(CH₃)₂ | H | H | H |
| DHP-341 | C₁₀H₂₁ | CH₃ | CH₃ | C(O)OCH(CH₃)₂ | C(O)OCH₂CH₃ | OCH(CH₃)₂ | H | H | H |
| DHP-342 | C₁₀H₂₁ | CH₃ | CH₃ | C(O)OCH(CH₃)₂ | C(O)OCH(CH₃)₂ | OCH(CH₃)₂ | H | H | H |
| DHP-343 | C₁₀H₂₁ | CH₃ | CH₃ | C(O)OCH₂CH₃ | C(O)OCH₂CH₃ | OCH₃ | H | H | H |
| DHP-344 | C₁₀H₂₁ | CH₃ | CH₃ | C(O)OCH₂CH₃ | C(O)OCH₂CH₃ | OCH₂CH₃ | H | H | H |
| DHP-345 | C₁₀H₂₁ | CH₃ | CH₃ | C(O)OCH₃ | C(O)OCH₃ | OCH₂CH₃ | H | H | H |
| DHP-346 | C₁₀H₂₁ | CH₃ | CH₃ | C(O)OCH₃ | C(O)OCH₃ | OCH₃ | H | H | H |
| DHP-347 | C₁₀H₂₁ | CH₃ | A* | C(O)OCH₂CH₃ | C(O)OCH₂CH₃ | OCH₃ | H | H | H |
| DHP-348 | C₁₀H₂₁ | CH₃ | A | C(O)OCH₂CH₃ | C(O)OCH₃ | OCH₃ | H | H | H |
| DHP-349 | C₁₀H₂₁ | CH₃ | A | C(O)OCH₂CH₃ | C(O)OCH(CH₃)₂ | OCH₃ | H | H | H |
| DHP-350 | C₁₀H₂₁ | CH₃ | A | C(O)OCH₂CH₃ | C(O)OCH₂CH₃ | OCH(CH₃)₂ | H | H | H |
| DHP-351 | C₁₀H₂₁ | CH₃ | A | C(O)OCH₂CH₃ | C(O)OCH₃ | OCH(CH₃)₂ | H | H | H |
| DHP-352 | C₁₀H₂₁ | CH₃ | A | C(O)OCH₂CH₃ | C(O)OCH(CH₃)₂ | OCH(CH₃)₂ | H | H | H |
| DHP-353 | C₁₀H₂₁ | CH₃ | A | C(O)OCH₂CH₃ | C(O)OCH₂CH₃ | OCH₂CH₃ | H | H | H |
| DHP-354 | C₁₀H₂₁ | CH₃ | A | C(O)OCH₂CH₃ | C(O)OCH₃ | OCH₂CH₃ | H | H | H |
| DHP-355 | C₁₀H₂₁ | CH₃ | A | C(O)OCH₂CH₃ | C(O)OCH(CH₃)₂ | OCH₂CH₃ | H | H | H |
| DHP-356 | C₁₀H₂₁ | CH₃ | A | C(O)OCH₃ | C(O)OCH₃ | OCH₃ | H | H | H |
| DHP-357 | C₁₀H₂₁ | CH₃ | A | C(O)OCH₃ | C(O)OCH₂CH₃ | OCH₃ | H | H | H |
| DHP-358 | C₁₀H₂₁ | CH₃ | A | C(O)OCH₃ | C(O)OCH(CH₃)₂ | OCH₃ | H | H | H |
| DHP-359 | C₁₀H₂₁ | CH₃ | A | C(O)OCH₃ | C(O)OCH₃ | OCH(CH₃)₂ | H | H | H |
| DHP-360 | C₁₀H₂₁ | CH₃ | A | C(O)OCH₃ | C(O)OCH₂CH₃ | OCH(CH₃)₂ | H | H | H |
| DHP-361 | C₁₀H₂₁ | CH₃ | A | C(O)OCH₃ | C(O)OCH(CH₃)₂ | OCH(CH₃)₂ | H | H | H |
| DHP-362 | C₁₀H₂₁ | CH₃ | A | C(O)OCH₃ | C(O)OCH₃ | OCH₂CH₃ | H | H | H |
| DHP-363 | C₁₀H₂₁ | CH₃ | A | C(O)OCH₃ | C(O)OCH₂CH₃ | OCH₂CH₃ | H | H | H |
| DHP-364 | C₁₀H₂₁ | CH₃ | A | C(O)OCH₃ | C(O)OCH(CH₃)₂ | OCH₂CH₃ | H | H | H |
| DHP-365 | C₁₀H₂₁ | CH₃ | A | C(O)OCH(CH₃)₂ | C(O)OCH(CH₃)₂ | OCH₃ | H | H | H |
| DHP-366 | C₁₀H₂₁ | CH₃ | A | C(O)OCH(CH₃)₂ | C(O)OCH₃ | OCH₃ | H | H | H |
| DHP-367 | C₁₀H₂₁ | CH₃ | A | C(O)OCH(CH₃)₂ | C(O)OCH₂CH₃ | OCH₃ | H | H | H |
| DHP-368 | C₁₀H₂₁ | CH₃ | A | C(O)OCH(CH₃)₂ | C(O)OCH(CH₃)₂ | OCH(CH₃)₂ | H | H | H |
| DHP-369 | C₁₀H₂₁ | CH₃ | A | C(O)OCH(CH₃)₂ | C(O)OCH₃ | OCH(CH₃)₂ | H | H | H |
| DHP-370 | C₁₀H₂₁ | CH₃ | A | C(O)OCH(CH₃)₂ | C(O)OCH₂CH₃ | OCH(CH₃)₂ | H | H | H |
| DHP-371 | C₁₀H₂₁ | CH₃ | A | C(O)OCH(CH₃)₂ | C(O)OCH(CH₃)₂ | OCH₂CH₃ | H | H | H |
| DHP-372 | C₁₀H₂₁ | CH₃ | A | C(O)OCH(CH₃)₂ | C(O)OCH₃ | OCH₂CH₃ | H | H | H |
| DHP-373 | C₁₀H₂₁ | CH₃ | A | C(O)OCH(CH₃)₂ | C(O)OCH₂CH₃ | OCH₂CH₃ | H | H | H |
| DHP-374 | C₁₀H₂₁ | CH₃ | A | C(O)OH | C(O)OH | OCH₃ | H | H | H |
| DHP-375 | C₁₀H₂₁ | CH₃ | A | C(O)OH | C(O)OCH₃ | OCH₃ | H | H | H |
| DHP-376 | C₁₀H₂₁ | CH₃ | A | C(O)OH | C(O)OCH₂CH₃ | OCH₃ | H | H | H |
| DHP-377 | C₁₀H₂₁ | CH₃ | A | C(O)OH | C(O)OCH(CH₃)₂ | OCH₃ | H | H | H |
| DHP-378 | C₁₀H₂₁ | CH₃ | A | C(O)OH | C(O)OH | OCH(CH₃)₂ | H | H | H |
| DHP-379 | C₁₀H₂₁ | CH₃ | A | C(O)OH | C(O)OCH₃ | OCH(CH₃)₂ | H | H | H |
| DHP-380 | C₁₀H₂₁ | CH₃ | A | C(O)OH | C(O)OCH₂CH₃ | OCH(CH₃)₂ | H | H | H |
| DHP-381 | C₁₀H₂₁ | CH₃ | A | C(O)OH | C(O)OCH(CH₃)₂ | OCH(CH₃)₂ | H | H | H |
| DHP-382 | C₁₀H₂₁ | CH₃ | A | C(O)OH | C(O)OH | OCH₂CH₃ | H | H | H |
| DHP-383 | C₁₀H₂₁ | CH₃ | A | C(O)OH | C(O)OCH₃ | OCH₂CH₃ | H | H | H |
| DHP-384 | C₁₀H₂₁ | CH₃ | A | C(O)OH | C(O)OCH₂CH₃ | OCH₂CH₃ | H | H | H |
| DHP-385 | C₁₀H₂₁ | CH₃ | A | C(O)OH | C(O)OCH(CH₃)₂ | OCH₂CH₃ | H | H | H |
| DHP-386 | C₁₀H₂₁ | CH₃ | A | C(O)OCH₃ | C(O)OH | OCH₃ | H | H | H |
| DHP-387 | C₁₀H₂₁ | CH₃ | A | C(O)OCH₂CH₃ | C(O)OH | OCH₃ | H | H | H |
| DHP-388 | C₁₀H₂₁ | CH₃ | A | C(O)OCH(CH₃)₂ | C(O)OH | OCH₃ | H | H | H |
| DHP-389 | C₁₀H₂₁ | CH₃ | A | C(O)OCH₃ | C(O)OH | OCH(CH₃)₂ | H | H | H |
| DHP-390 | C₁₀H₂₁ | CH₃ | A | C(O)OCH₂CH₃ | C(O)OH | OCH(CH₃)₂ | H | H | H |
| DHP-391 | C₁₀H₂₁ | CH₃ | A | C(O)OCH(CH₃)₂ | C(O)OH | OCH(CH₃)₂ | H | H | H |
| DHP-392 | C₁₀H₂₁ | CH₃ | A | C(O)OCH₃ | C(O)OH | OCH₂CH₃ | H | H | H |
| DHP-393 | C₁₀H₂₁ | CH₃ | A | C(O)OCH₂CH₃ | C(O)OH | OCH₂CH₃ | H | H | H |
| DHP-394 | C₁₀H₂₁ | CH₃ | A | C(O)OCH(CH₃)₂ | C(O)OH | OCH₂CH₃ | H | H | H |
| DHP-395 | C₁₀H₂₁ | CH₃ | CH₃ | C(O)OCH₂CH₂OCH₃ | C(O)OCH₂CH₃ | OCH₂CH₃ | H | H | H |
| DHP-396 | C₁₀H₂₁ | CH₃ | CH₃ | C(O)OCH₂CH₂OCH₃ | C(O)OCH₃ | OCH₂CH₃ | H | H | H |
| DHP-397 | C₁₀H₂₁ | CH₃ | CH₃ | C(O)OCH₂CH₂OCH₃ | C(O)OCH(CH₃)₂ | OCH₂CH₃ | H | H | H |
| DHP-398 | C₁₀H₂₁ | CH₃ | CH₃ | C(O)OCH₂CH₂OCH₃ | C(O)OCH₂CH₃ | OCH(CH₃)₂ | H | H | H |
| DHP-399 | C₁₀H₂₁ | CH₃ | CH₃ | C(O)OCH₂CH₂OCH₃ | C(O)OCH₃ | OCH(CH₃)₂ | H | H | H |
| DHP-400 | C₁₀H₂₁ | CH₃ | CH₃ | C(O)OCH₂CH₂OCH₃ | C(O)OCH(CH₃)₂ | OCH(CH₃)₂ | H | H | H |
| DHP-401 | C₁₀H₂₁ | CH₃ | CH₃ | C(O)OCH₂CH₂OCH₃ | C(O)OCH₂CH₃ | OCH₃ | H | H | H |
| DHP-402 | C₁₀H₂₁ | CH₃ | CH₃ | C(O)OCH₂CH₂OCH₃ | C(O)OCH₃ | OCH₃ | H | H | H |
| DHP-403 | C₁₀H₂₁ | CH₃ | CH₃ | C(O)OCH₂CH₂OCH₃ | C(O)OCH(CH₃)₂ | OCH₃ | H | H | H |
| DHP-404 | C₁₀H₂₁ | B** | CH₃ | C(O)OCH₂CH₃ | C(O)OCH₂CH₃ | OCH₃ | H | H | H |
| DHP-405 | C₁₀H₂₁ | B | CH₃ | C(O)OCH₂CH₃ | C(O)OCH₃ | OCH₃ | H | H | H |
| DHP-406 | C₁₀H₂₁ | B | CH₃ | C(O)OCH₂CH₃ | C(O)OCH(CH₃)₂ | OCH₃ | H | H | H |
| DHP-407 | C₁₀H₂₁ | B | CH₃ | C(O)OCH₂CH₃ | C(O)OCH₂CH₃ | OCH(CH₃)₂ | H | H | H |
| DHP-408 | C₁₀H₂₁ | B | CH₃ | C(O)OCH₂CH₃ | C(O)OCH₃ | OCH(CH₃)₂ | H | H | H |
| DHP-409 | C₁₀H₂₁ | B | CH₃ | C(O)OCH₂CH₃ | C(O)OCH(CH₃)₂ | OCH(CH₃)₂ | H | H | H |
| DHP-410 | C₁₀H₂₁ | B | CH₃ | C(O)OCH₂CH₃ | C(O)OCH₂CH₃ | OCH₂CH₃ | H | H | H |
| DHP-411 | C₁₀H₂₁ | B | CH₃ | C(O)OCH₂CH₃ | C(O)OCH₃ | OCH₂CH₃ | H | H | H |
| DHP-412 | C₁₀H₂₁ | B | CH₃ | C(O)OCH₂CH₃ | C(O)OCH(CH₃)₂ | OCH₂CH₃ | H | H | H |
| DHP-413 | C₁₀H₂₁ | B | CH₃ | C(O)OCH₃ | C(O)OCH₃ | OCH₃ | H | H | H |
| DHP-414 | C₁₀H₂₁ | B | CH₃ | C(O)OCH₃ | C(O)OCH₂CH₃ | OCH₃ | H | H | H |
| DHP-415 | C₁₀H₂₁ | B | CH₃ | C(O)OCH₃ | C(O)OCH(CH₃)₂ | OCH₃ | H | H | H |
| DHP-416 | C₁₀H₂₁ | B | CH₃ | C(O)OCH₃ | C(O)OCH₃ | OCH(CH₃)₂ | H | H | H |
| DHP-417 | C₁₀H₂₁ | B | CH₃ | C(O)OCH₃ | C(O)OCH₂CH₃ | OCH(CH₃)₂ | H | H | H |
| DHP-418 | C₁₀H₂₁ | B | CH₃ | C(O)OCH₃ | C(O)OCH(CH₃)₂ | OCH(CH₃)₂ | H | H | H |
| DHP-419 | C₁₀H₂₁ | B | CH₃ | C(O)OCH₃ | C(O)OCH₃ | OCH₂CH₃ | H | H | H |
| DHP-420 | C₁₀H₂₁ | B | CH₃ | C(O)OCH₃ | C(O)OCH₂CH₃ | OCH₂CH₃ | H | H | H |
| DHP-421 | C₁₀H₂₁ | B | CH₃ | C(O)OCH₃ | C(O)OCH(CH₃)₂ | OCH₂CH₃ | H | H | H |
| DHP-422 | C₁₀H₂₁ | B | CH₃ | C(O)OCH(CH₃)₂ | C(O)OCH(CH₃)₂ | OCH₃ | H | H | H |
| DHP-423 | C₁₀H₂₁ | B | CH₃ | C(O)OCH(CH₃)₂ | C(O)OCH₃ | OCH₃ | H | H | H |
| DHP-424 | C₁₀H₂₁ | B | CH₃ | C(O)OCH(CH₃)₂ | C(O)OCH₂CH₃ | OCH₃ | H | H | H |
| DHP-425 | C₁₀H₂₁ | B | CH₃ | C(O)OCH(CH₃)₂ | C(O)OCH(CH₃)₂ | OCH(CH₃)₂ | H | H | H |
| DHP-426 | C₁₀H₂₁ | B | CH₃ | C(O)OCH(CH₃)₂ | C(O)OCH₃ | OCH(CH₃)₂ | H | H | H |
| DHP-427 | C₁₀H₂₁ | B | CH₃ | C(O)OCH(CH₃)₂ | C(O)OCH₂CH₃ | OCH(CH₃)₂ | H | H | H |
| DHP-428 | C₁₀H₂₁ | B | CH₃ | C(O)OCH(CH₃)₂ | C(O)OCH(CH₃)₂ | OCH₂CH₃ | H | H | H |
| DHP-429 | C₁₀H₂₁ | B | CH₃ | C(O)OCH(CH₃)₂ | C(O)OCH₃ | OCH₂CH₃ | H | H | H |
| DHP-430 | C₁₀H₂₁ | B | CH₃ | C(O)OCH(CH₃)₂ | C(O)OCH₂CH₃ | OCH₂CH₃ | H | H | H |
| DHP-431 | C₁₀H₂₁ | B | CH₃ | C(O)OH | C(O)OH | OCH₃ | H | H | H |
| DHP-432 | C₁₀H₂₁ | B | CH₃ | C(O)OH | C(O)OCH₃ | OCH₃ | H | H | H |
| DHP-433 | C₁₀H₂₁ | B | CH₃ | C(O)OH | C(O)OCH₂CH₃ | OCH₃ | H | H | H |
| DHP-434 | C₁₀H₂₁ | B | CH₃ | C(O)OH | C(O)OCH(CH₃)₂ | OCH₃ | H | H | H |
| DHP-435 | C₁₀H₂₁ | B | CH₃ | C(O)OH | C(O)OH | OCH(CH₃)₂ | H | H | H |
| DHP-436 | C₁₀H₂₁ | B | CH₃ | C(O)OH | C(O)OCH₃ | OCH(CH₃)₂ | H | H | H |
| DHP-437 | C₁₀H₂₁ | B | CH₃ | C(O)OH | C(O)OCH₂CH₃ | OCH(CH₃)₂ | H | H | H |
| DHP-438 | C₁₀H₂₁ | B | CH₃ | C(O)OH | C(O)OCH(CH₃)₂ | OCH(CH₃)₂ | H | H | H |
| DHP-439 | C₁₀H₂₁ | B | CH₃ | C(O)OH | C(O)OH | OCH₂CH₃ | H | H | H |
| DHP-440 | C₁₀H₂₁ | B | CH₃ | C(O)OH | C(O)OCH₃ | OCH₂CH₃ | H | H | H |
| DHP-441 | C₁₀H₂₁ | B | CH₃ | C(O)OH | C(O)OCH₂CH₃ | OCH₂CH₃ | H | H | H |
| DHP-442 | C₁₀H₂₁ | B | CH₃ | C(O)OH | C(O)OCH(CH₃)₂ | OCH₂CH₃ | H | H | H |
| DHP-443 | C₁₀H₂₁ | B | CH₃ | C(O)OCH₃ | C(O)OH | OCH₃ | H | H | H |
| DHP-444 | C₁₀H₂₁ | B | CH₃ | C(O)OCH₂CH₃ | C(O)OH | OCH₃ | H | H | H |
| DHP-445 | C₁₀H₂₁ | B | CH₃ | C(O)OCH(CH₃)₂ | C(O)OH | OCH₃ | H | H | H |
| DHP-446 | C₁₀H₂₁ | B | CH₃ | C(O)OCH₃ | C(O)OH | OCH(CH₃)₂ | H | H | H |
| DHP-447 | C₁₀H₂₁ | B | CH₃ | C(O)OCH₂CH₃ | C(O)OH | OCH(CH₃)₂ | H | H | H |
| DHP-448 | C₁₀H₂₁ | B | CH₃ | C(O)OCH(CH₃)₂ | C(O)OH | OCH(CH₃)₂ | H | H | H |
| DHP-449 | C₁₀H₂₁ | B | CH₃ | C(O)OCH₃ | C(O)OH | OCH₂CH₃ | H | H | H |
| DHP-450 | C₁₀H₂₁ | B | CH₃ | C(O)OCH₂CH₃ | C(O)OH | OCH₂CH₃ | H | H | H |
| DHP-451 | C₁₀H₂₁ | B | CH₃ | C(O)OCH(CH₃)₂ | C(O)OH | OCH₂CH₃ | H | H | H |
| DHP-452 | C₁₀H₂₁ | CH₃ | CH₃ | C(O)OCH₂CH₃ | C(O)OCH₂CH₃ | OCH₃ | NO₂ | NO₂ | H |
| DHP-453 | C₁₀H₂₁ | CH₃ | CH₃ | C(O)OCH₂CH₃ | C(O)OCH₃ | OCH₃ | NO₂ | NO₂ | H |
| DHP-454 | C₁₀H₂₁ | CH₃ | CH₃ | C(O)OCH₂CH₃ | C(O)OCH(CH₃)₂ | OCH₃ | NO₂ | NO₂ | H |
| DHP-455 | C₁₀H₂₁ | CH₃ | CH₃ | C(O)OCH₂CH₃ | C(O)OCH₂CH₃ | OCH(CH₃)₂ | NO₂ | NO₂ | H |
| DHP-456 | C₁₀H₂₁ | CH₃ | CH₃ | C(O)OCH₂CH₃ | C(O)OCH₃ | OCH(CH₃)₂ | NO₂ | NO₂ | H |
| DHP-457 | C₁₀H₂₁ | CH₃ | CH₃ | C(O)OCH₂CH₃ | C(O)OCH(CH₃)₂ | OCH(CH₃)₂ | NO₂ | NO₂ | H |
| DHP-458 | C₁₀H₂₁ | CH₃ | CH₃ | C(O)OCH₂CH₃ | C(O)OCH₂CH₃ | OCH₂CH₃ | NO₂ | NO₂ | H |
| DHP-459 | C₁₀H₂₁ | CH₃ | CH₃ | C(O)OCH₂CH₃ | C(O)OCH₃ | OCH₂CH₃ | NO₂ | NO₂ | H |
| DHP-460 | C₁₀H₂₁ | CH₃ | CH₃ | C(O)OCH₂CH₃ | C(O)OCH(CH₃)₂ | OCH₂CH₃ | NO₂ | NO₂ | H |
| DHP-461 | C₁₀H₂₁ | CH₃ | CH₃ | C(O)OCH₃ | C(O)OCH₃ | OCH₃ | NO₂ | NO₂ | H |
| DHP-462 | C₁₀H₂₁ | CH₃ | CH₃ | C(O)OCH₃ | C(O)OCH₂CH₃ | OCH₃ | NO₂ | NO₂ | H |
| DHP-463 | C₁₀H₂₁ | CH₃ | CH₃ | C(O)OCH₃ | C(O)OCH(CH₃)₂ | OCH₃ | NO₂ | NO₂ | H |
| DHP-464 | C₁₀H₂₁ | CH₃ | CH₃ | C(O)OCH₃ | C(O)OCH₃ | OCH(CH₃)₂ | NO₂ | NO₂ | H |
| DHP-465 | C₁₀H₂₁ | CH₃ | CH₃ | C(O)OCH₃ | C(O)OCH₂CH₃ | OCH(CH₃)₂ | NO₂ | NO₂ | H |
| DHP-466 | C₁₀H₂₁ | CH₃ | CH₃ | C(O)OCH₃ | C(O)OCH(CH₃)₂ | OCH(CH₃)₂ | NO₂ | NO₂ | H |
| DHP-467 | C₁₀H₂₁ | CH₃ | CH₃ | C(O)OCH₃ | C(O)OCH₃ | OCH₂CH₃ | NO₂ | NO₂ | H |
| DHP-468 | C₁₀H₂₁ | CH₃ | CH₃ | C(O)OCH₃ | C(O)OCH₂CH₃ | OCH₂CH₃ | NO₂ | NO₂ | H |
| DHP-469 | C₁₀H₂₁ | CH₃ | CH₃ | C(O)OCH₃ | C(O)OCH(CH₃)₂ | OCH₂CH₃ | NO₂ | NO₂ | H |
| DHP-470 | C₁₀H₂₁ | CH₃ | CH₃ | C(O)OCH(CH₃)₂ | C(O)OCH(CH₃)₂ | OCH₃ | NO₂ | NO₂ | H |
| DHP-471 | C₁₀H₂₁ | CH₃ | CH₃ | C(O)OCH(CH₃)₂ | C(O)OCH₃ | OCH₃ | NO₂ | NO₂ | H |
| DHP-472 | C₁₀H₂₁ | CH₃ | CH₃ | C(O)OCH(CH₃)₂ | C(O)OCH₂CH₃ | OCH₃ | NO₂ | NO₂ | H |
| DHP-473 | C₁₀H₂₁ | CH₃ | CH₃ | C(O)OCH(CH₃)₂ | C(O)OCH(CH₃)₂ | OCH(CH₃)₂ | NO₂ | NO₂ | H |
| DHP-474 | C₁₀H₂₁ | CH₃ | CH₃ | C(O)OCH(CH₃)₂ | C(O)OCH₃ | OCH(CH₃)₂ | NO₂ | NO₂ | H |
| DHP-475 | C₁₀H₂₁ | CH₃ | CH₃ | C(O)OCH(CH₃)₂ | C(O)OCH₂CH₃ | OCH(CH₃)₂ | NO₂ | NO₂ | H |
| DHP-476 | C₁₀H₂₁ | CH₃ | CH₃ | C(O)OCH(CH₃)₂ | C(O)OCH(CH₃)₂ | OCH₂CH₃ | NO₂ | NO₂ | H |
| DHP-477 | C₁₀H₂₁ | CH₃ | CH₃ | C(O)OCH(CH₃)₂ | C(O)OCH₃ | OCH₂CH₃ | NO₂ | NO₂ | H |
| DHP-478 | C₁₀H₂₁ | CH₃ | CH₃ | C(O)OCH(CH₃)₂ | C(O)OCH₂CH₃ | OCH₂CH₃ | NO₂ | NO₂ | H |
| DHP-479 | C₁₀H₂₁ | CH₃ | CH₃ | C(O)OH | C(O)OH | OCH₃ | NO₂ | NO₂ | H |
| DHP-480 | C₁₀H₂₁ | CH₃ | CH₃ | C(O)OH | C(O)OCH₃ | OCH₃ | NO₂ | NO₂ | H |
| DHP-481 | C₁₀H₂₁ | CH₃ | CH₃ | C(O)OH | C(O)OCH₂CH₃ | OCH₃ | NO₂ | NO₂ | H |
| DHP-482 | C₁₀H₂₁ | CH₃ | CH₃ | C(O)OH | C(O)OCH(CH₃)₂ | OCH₃ | NO₂ | NO₂ | H |
| DHP-483 | C₁₀H₂₁ | CH₃ | CH₃ | C(O)OH | C(O)OH | OCH(CH₃)₂ | NO₂ | NO₂ | H |
| DHP-484 | C₁₀H₂₁ | CH₃ | CH₃ | C(O)OH | C(O)OCH₃ | OCH(CH₃)₂ | NO₂ | NO₂ | H |
| DHP-485 | C₁₀H₂₁ | CH₃ | CH₃ | C(O)OH | C(O)OCH₂CH₃ | OCH(CH₃)₂ | NO₂ | NO₂ | H |
| DHP-486 | C₁₀H₂₁ | CH₃ | CH₃ | C(O)OH | C(O)OCH(CH₃)₂ | OCH(CH₃)₂ | NO₂ | NO₂ | H |
| DHP-487 | C₁₀H₂₁ | CH₃ | CH₃ | C(O)OH | C(O)OH | OCH₂CH₃ | NO₂ | NO₂ | H |
| DHP-488 | C₁₀H₂₁ | CH₃ | CH₃ | C(O)OH | C(O)OCH₃ | OCH₂CH₃ | NO₂ | NO₂ | H |
| DHP-489 | C₁₀H₂₁ | CH₃ | CH₃ | C(O)OH | C(O)OCH₂CH₃ | OCH₂CH₃ | NO₂ | NO₂ | H |
| DHP-490 | C₁₀H₂₁ | CH₃ | CH₃ | C(O)OH | C(O)OCH(CH₃)₂ | OCH₂CH₃ | NO₂ | NO₂ | H |
| DHP-491 | C₁₀H₂₁ | CH₃ | CH₃ | C(O)OCH₃ | C(O)OH | OCH₃ | NO₂ | NO₂ | H |
| DHP-492 | C₁₀H₂₁ | CH₃ | CH₃ | C(O)OCH₂CH₃ | C(O)OH | OCH₃ | NO₂ | NO₂ | H |
| DHP-493 | C₁₀H₂₁ | CH₃ | CH₃ | C(O)OCH(CH₃)₂ | C(O)OH | OCH₃ | NO₂ | NO₂ | H |
| DHP-494 | C₁₀H₂₁ | CH₃ | CH₃ | C(O)OCH₃ | C(O)OH | OCH(CH₃)₂ | NO₂ | NO₂ | H |
| DHP-495 | C₁₀H₂₁ | CH₃ | CH₃ | C(O)OCH₂CH₃ | C(O)OH | OCH(CH₃)₂ | NO₂ | NO₂ | H |
| DHP-496 | C₁₀H₂₁ | CH₃ | CH₃ | C(O)OCH(CH₃)₂ | C(O)OH | OCH(CH₃)₂ | NO₂ | NO₂ | H |
| DHP-497 | C₁₀H₂₁ | CH₃ | CH₃ | C(O)OCH₃ | C(O)OH | OCH₂CH₃ | NO₂ | NO₂ | H |
| DHP-498 | C₁₀H₂₁ | CH₃ | CH₃ | C(O)OCH₂CH₃ | C(O)OH | OCH₂CH₃ | NO₂ | NO₂ | H |
| DHP-499 | C₁₀H₂₁ | CH₃ | CH₃ | C(O)OCH(CH₃)₂ | C(O)OH | OCH₂CH₃ | NO₂ | NO₂ | H |
| DHP-500 | C₁₀H₂₁ | CH₃ | CH₃ | C(O)OCH₂CH₃ | C(O)OCH₂CH₃ | OCH₃ | NO₂ | H | H |
| DHP-501 | C₁₀H₂₁ | CH₃ | CH₃ | C(O)OCH₂CH₃ | C(O)OCH₃ | OCH₃ | NO₂ | H | H |
| DHP-502 | C₁₀H₂₁ | CH₃ | CH₃ | C(O)OCH₂CH₃ | C(O)OCH(CH₃)₂ | OCH₃ | NO₂ | H | H |
| DHP-503 | C₁₀H₂₁ | CH₃ | CH₃ | C(O)OCH₂CH₃ | C(O)OCH₂CH₃ | OCH(CH₃)₂ | NO₂ | H | H |
| DHP-504 | C₁₀H₂₁ | CH₃ | CH₃ | C(O)OCH₂CH₃ | C(O)OCH₃ | OCH(CH₃)₂ | NO₂ | H | H |
| DHP-505 | C₁₀H₂₁ | CH₃ | CH₃ | C(O)OCH₂CH₃ | C(O)OCH(CH₃)₂ | OCH(CH₃)₂ | NO₂ | H | H |
| DHP-506 | C₁₀H₂₁ | CH₃ | CH₃ | C(O)OCH₂CH₃ | C(O)OCH₂CH₃ | OCH₂CH₃ | NO₂ | H | H |
| DHP-507 | C₁₀H₂₁ | CH₃ | CH₃ | C(O)OCH₂CH₃ | C(O)OCH₃ | OCH₂CH₃ | NO₂ | H | H |
| DHP-508 | C₁₀H₂₁ | CH₃ | CH₃ | C(O)OCH₂CH₃ | C(O)OCH(CH₃)₂ | OCH₂CH₃ | NO₂ | H | H |
| DHP-509 | C₁₀H₂₁ | CH₃ | CH₃ | C(O)OCH₃ | C(O)OCH₃ | OCH₃ | NO₂ | H | H |
| DHP-510 | C₁₀H₂₁ | CH₃ | CH₃ | C(O)OCH₃ | C(O)OCH₂CH₃ | OCH₃ | NO₂ | H | H |
| DHP-511 | C₁₀H₂₁ | CH₃ | CH₃ | C(O)OCH₃ | C(O)OCH(CH₃)₂ | OCH₃ | NO₂ | H | H |
| DHP-512 | C₁₀H₂₁ | CH₃ | CH₃ | C(O)OCH₃ | C(O)OCH₃ | OCH(CH₃)₂ | NO₂ | H | H |
| DHP-513 | C₁₀H₂₁ | CH₃ | CH₃ | C(O)OCH₃ | C(O)OCH₂CH₃ | OCH(CH₃)₂ | NO₂ | H | H |
| DHP-514 | C₁₀H₂₁ | CH₃ | CH₃ | C(O)OCH₃ | C(O)OCH(CH₃)₂ | OCH(CH₃)₂ | NO₂ | H | H |
| DHP-515 | C₁₀H₂₁ | CH₃ | CH₃ | C(O)OCH₃ | C(O)OCH₃ | OCH₂CH₃ | NO₂ | H | H |
| DHP-516 | C₁₀H₂₁ | CH₃ | CH₃ | C(O)OCH₃ | C(O)OCH₂CH₃ | OCH₂CH₃ | NO₂ | H | H |
| DHP-517 | C₁₀H₂₁ | CH₃ | CH₃ | C(O)OCH₃ | C(O)OCH(CH₃)₂ | OCH₂CH₃ | NO₂ | H | H |
| DHP-518 | C₁₀H₂₁ | CH₃ | CH₃ | C(O)OCH(CH₃)₂ | C(O)OCH(CH₃)₂ | OCH₃ | NO₂ | H | H |
| DHP-519 | C₁₀H₂₁ | CH₃ | CH₃ | C(O)OCH(CH₃)₂ | C(O)OCH₃ | OCH₃ | NO₂ | H | H |
| DHP-520 | C₁₀H₂₁ | CH₃ | CH₃ | C(O)OCH(CH₃)₂ | C(O)OCH₂CH₃ | OCH₃ | NO₂ | H | H |
| DHP-521 | C₁₀H₂₁ | CH₃ | CH₃ | C(O)OCH(CH₃)₂ | C(O)OCH(CH₃)₂ | OCH(CH₃)₂ | NO₂ | H | H |
| DHP-522 | C₁₀H₂₁ | CH₃ | CH₃ | C(O)OCH(CH₃)₂ | C(O)OCH₃ | OCH(CH₃)₂ | NO₂ | H | H |
| DHP-523 | C₁₀H₂₁ | CH₃ | CH₃ | C(O)OCH(CH₃)₂ | C(O)OCH₂CH₃ | OCH(CH₃)₂ | NO₂ | H | H |
| DHP-524 | C₁₀H₂₁ | CH₃ | CH₃ | C(O)OCH(CH₃)₂ | C(O)OCH(CH₃)₂ | OCH₂CH₃ | NO₂ | H | H |
| DHP-525 | C₁₀H₂₁ | CH₃ | CH₃ | C(O)OCH(CH₃)₂ | C(O)OCH₃ | OCH₂CH₃ | NO₂ | H | H |
| DHP-526 | C₁₀H₂₁ | CH₃ | CH₃ | C(O)OCH(CH₃)₂ | C(O)OCH₂CH₃ | OCH₂CH₃ | NO₂ | H | H |
| DHP-527 | C₁₀H₂₁ | CH₃ | CH₃ | C(O)OH | C(O)OH | OCH₃ | NO₂ | H | H |
| DHP-528 | C₁₀H₂₁ | CH₃ | CH₃ | C(O)OH | C(O)OCH₃ | OCH₃ | NO₂ | H | H |
| DHP-529 | C₁₀H₂₁ | CH₃ | CH₃ | C(O)OH | C(O)OCH₂CH₃ | OCH₃ | NO₂ | H | H |
| DHP-530 | C₁₀H₂₁ | CH₃ | CH₃ | C(O)OH | C(O)OCH(CH₃)₂ | OCH₃ | NO₂ | H | H |
| DHP-531 | C₁₀H₂₁ | CH₃ | CH₃ | C(O)OH | C(O)OH | OCH(CH₃)₂ | NO₂ | H | H |
| DHP-532 | C₁₀H₂₁ | CH₃ | CH₃ | C(O)OH | C(O)OCH₃ | OCH(CH₃)₂ | NO₂ | H | H |
| DHP-533 | C₁₀H₂₁ | CH₃ | CH₃ | C(O)OH | C(O)OCH₂CH₃ | OCH(CH₃)₂ | NO₂ | H | H |
| DHP-534 | C₁₀H₂₁ | CH₃ | CH₃ | C(O)OH | C(O)OCH(CH₃)₂ | OCH(CH₃)₂ | NO₂ | H | H |
| DHP-535 | C₁₀H₂₁ | CH₃ | CH₃ | C(O)OH | C(O)OH | OCH₂CH₃ | NO₂ | H | H |
| DHP-536 | C₁₀H₂₁ | CH₃ | CH₃ | C(O)OH | C(O)OCH₃ | OCH₂CH₃ | NO₂ | H | H |
| DHP-537 | C₁₀H₂₁ | CH₃ | CH₃ | C(O)OH | C(O)OCH₂CH₃ | OCH₂CH₃ | NO₂ | H | H |
| DHP-538 | C₁₀H₂₁ | CH₃ | CH₃ | C(O)OH | C(O)OCH(CH₃)₂ | OCH₂CH₃ | NO₂ | H | H |
| DHP-539 | C₁₀H₂₁ | CH₃ | CH₃ | C(O)OCH₃ | C(O)OH | OCH₃ | NO₂ | H | H |
| DHP-540 | C₁₀H₂₁ | CH₃ | CH₃ | C(O)OCH₂CH₃ | C(O)OH | OCH₃ | NO₂ | H | H |
| DHP-541 | C₁₀H₂₁ | CH₃ | CH₃ | C(O)OCH(CH₃)₂ | C(O)OH | OCH₃ | NO₂ | H | H |
| DHP-542 | C₁₀H₂₁ | CH₃ | CH₃ | C(O)OCH₃ | C(O)OH | OCH(CH₃)₂ | NO₂ | H | H |
| DHP-543 | C₁₀H₂₁ | CH₃ | CH₃ | C(O)OCH₂CH₃ | C(O)OH | OCH(CH₃)₂ | NO₂ | H | H |
| DHP-544 | C₁₀H₂₁ | CH₃ | CH₃ | C(O)OCH(CH₃)₂ | C(O)OH | OCH(CH₃)₂ | NO₂ | H | H |
| DHP-545 | C₁₀H₂₁ | CH₃ | CH₃ | C(O)OCH₃ | C(O)OH | OCH₂CH₃ | NO₂ | H | H |
| DHP-546 | C₁₀H₂₁ | CH₃ | CH₃ | C(O)OCH₂CH₃ | C(O)OH | OCH₂CH₃ | NO₂ | H | H |
| DHP-547 | C₁₀H₂₁ | CH₃ | CH₃ | C(O)OCH(CH₃)₂ | C(O)OH | OCH₂CH₃ | NO₂ | H | H |
| DHP-548 | C₁₀H₂₁ | CH₃ | CH₃ | C(O)OCH₂CH₃ | C(O)OCH₂CH₃ | OCH₃ | H | NO₂ | H |
| DHP-549 | C₁₀H₂₁ | CH₃ | CH₃ | C(O)OCH₂CH₃ | C(O)OCH₃ | OCH₃ | H | NO₂ | H |
| DHP-550 | C₁₀H₂₁ | CH₃ | CH₃ | C(O)OCH₂CH₃ | C(O)OCH(CH₃)₂ | OCH₃ | H | NO₂ | H |
| DHP-551 | C₁₀H₂₁ | CH₃ | CH₃ | C(O)OCH₂CH₃ | C(O)OCH₂CH₃ | OCH(CH₃)₂ | H | NO₂ | H |
| DHP-552 | C₁₀H₂₁ | CH₃ | CH₃ | C(O)OCH₂CH₃ | C(O)OCH₃ | OCH(CH₃)₂ | H | NO₂ | H |
| DHP-553 | C₁₀H₂₁ | CH₃ | CH₃ | C(O)OCH₂CH₃ | C(O)OCH(CH₃)₂ | OCH(CH₃)₂ | H | NO₂ | H |
| DHP-554 | C₁₀H₂₁ | CH₃ | CH₃ | C(O)OCH₂CH₃ | C(O)OCH₂CH₃ | OCH₂CH₃ | H | NO₂ | H |
| DHP-555 | C₁₀H₂₁ | CH₃ | CH₃ | C(O)OCH₂CH₃ | C(O)OCH₃ | OCH₂CH₃ | H | NO₂ | H |
| DHP-556 | C₁₀H₂₁ | CH₃ | CH₃ | C(O)OCH₂CH₃ | C(O)OCH(CH₃)₂ | OCH₂CH₃ | H | NO₂ | H |
| DHP-557 | C₁₀H₂₁ | CH₃ | CH₃ | C(O)OCH₃ | C(O)OCH₃ | OCH₃ | H | NO₂ | H |
| DHP-558 | C₁₀H₂₁ | CH₃ | CH₃ | C(O)OCH₃ | C(O)OCH₂CH₃ | OCH₃ | H | NO₂ | H |
| DHP-559 | C₁₀H₂₁ | CH₃ | CH₃ | C(O)OCH₃ | C(O)OCH(CH₃)₂ | OCH₃ | H | NO₂ | H |
| DHP-560 | C₁₀H₂₁ | CH₃ | CH₃ | C(O)OCH₃ | C(O)OCH₃ | OCH(CH₃)₂ | H | NO₂ | H |
| DHP-561 | C₁₀H₂₁ | CH₃ | CH₃ | C(O)OCH₃ | C(O)OCH₂CH₃ | OCH(CH₃)₂ | H | NO₂ | H |
| DHP-562 | C₁₀H₂₁ | CH₃ | CH₃ | C(O)OCH₃ | C(O)OCH(CH₃)₂ | OCH(CH₃)₂ | H | NO₂ | H |
| DHP-563 | C₁₀H₂₁ | CH₃ | CH₃ | C(O)OCH₃ | C(O)OCH₃ | OCH₂CH₃ | H | NO₂ | H |
| DHP-564 | C₁₀H₂₁ | CH₃ | CH₃ | C(O)OCH₃ | C(O)OCH₂CH₃ | OCH₂CH₃ | H | NO₂ | H |
| DHP-565 | C₁₀H₂₁ | CH₃ | CH₃ | C(O)OCH₃ | C(O)OCH(CH₃)₂ | OCH₂CH₃ | H | NO₂ | H |
| DHP-566 | C₁₀H₂₁ | CH₃ | CH₃ | C(O)OCH(CH₃)₂ | C(O)OCH(CH₃)₂ | OCH₃ | H | NO₂ | H |
| DHP-567 | C₁₀H₂₁ | CH₃ | CH₃ | C(O)OCH(CH₃)₂ | C(O)OCH₃ | OCH₃ | H | NO₂ | H |
| DHP-568 | C₁₀H₂₁ | CH₃ | CH₃ | C(O)OCH(CH₃)₂ | C(O)OCH₂CH₃ | OCH₃ | H | NO₂ | H |
| DHP-569 | C₁₀H₂₁ | CH₃ | CH₃ | C(O)OCH(CH₃)₂ | C(O)OCH(CH₃)₂ | OCH(CH₃)₂ | H | NO₂ | H |
| DHP-570 | C₁₀H₂₁ | CH₃ | CH₃ | C(O)OCH(CH₃)₂ | C(O)OCH₃ | OCH(CH₃)₂ | H | NO₂ | H |
| DHP-571 | C₁₀H₂₁ | CH₃ | CH₃ | C(O)OCH(CH₃)₂ | C(O)OCH₂CH₃ | OCH(CH₃)₂ | H | NO₂ | H |
| DHP-572 | C₁₀H₂₁ | CH₃ | CH₃ | C(O)OCH(CH₃)₂ | C(O)OCH(CH₃)₂ | OCH₂CH₃ | H | NO₂ | H |
| DHP-573 | C₁₀H₂₁ | CH₃ | CH₃ | C(O)OCH(CH₃)₂ | C(O)OCH₃ | OCH₂CH₃ | H | NO₂ | H |
| DHP-574 | C₁₀H₂₁ | CH₃ | CH₃ | C(O)OCH(CH₃)₂ | C(O)OCH₂CH₃ | OCH₂CH₃ | H | NO₂ | H |
| DHP-575 | C₁₀H₂₁ | CH₃ | CH₃ | C(O)OH | C(O)OH | OCH₃ | H | NO₂ | H |
| DHP-576 | C₁₀H₂₁ | CH₃ | CH₃ | C(O)OH | C(O)OCH₃ | OCH₃ | H | NO₂ | H |
| DHP-577 | C₁₀H₂₁ | CH₃ | CH₃ | C(O)OH | C(O)OCH₂CH₃ | OCH₃ | H | NO₂ | H |
| DHP-578 | C₁₀H₂₁ | CH₃ | CH₃ | C(O)OH | C(O)OCH(CH₃)₂ | OCH₃ | H | NO₂ | H |
| DHP-579 | C₁₀H₂₁ | CH₃ | CH₃ | C(O)OH | C(O)OH | OCH(CH₃)₂ | H | NO₂ | H |
| DHP-580 | C₁₀H₂₁ | CH₃ | CH₃ | C(O)OH | C(O)OCH₃ | OCH(CH₃)₂ | H | NO₂ | H |
| DHP-581 | C₁₀H₂₁ | CH₃ | CH₃ | C(O)OH | C(O)OCH₂CH₃ | OCH(CH₃)₂ | H | NO₂ | H |
| DHP-582 | C₁₀H₂₁ | CH₃ | CH₃ | C(O)OH | C(O)OCH(CH₃)₂ | OCH(CH₃)₂ | H | NO₂ | H |
| DHP-583 | C₁₀H₂₁ | CH₃ | CH₃ | C(O)OH | C(O)OH | OCH₂CH₃ | H | NO₂ | H |
| DHP-584 | C₁₀H₂₁ | CH₃ | CH₃ | C(O)OH | C(O)OCH₃ | OCH₂CH₃ | H | NO₂ | H |
| DHP-585 | C₁₀H₂₁ | CH₃ | CH₃ | C(O)OH | C(O)OCH₂CH₃ | OCH₂CH₃ | H | NO₂ | H |
| DHP-586 | C₁₀H₂₁ | CH₃ | CH₃ | C(O)OH | C(O)OCH(CH₃)₂ | OCH₂CH₃ | H | NO₂ | H |
| DHP-587 | C₁₀H₂₁ | CH₃ | CH₃ | C(O)OCH₃ | C(O)OH | OCH₃ | H | NO₂ | H |
| DHP-588 | C₁₀H₂₁ | CH₃ | CH₃ | C(O)OCH₂CH₃ | C(O)OH | OCH₃ | H | NO₂ | H |
| DHP-589 | C₁₀H₂₁ | CH₃ | CH₃ | C(O)OCH(CH₃)₂ | C(O)OH | OCH₃ | H | NO₂ | H |
| DHP-590 | C₁₀H₂₁ | CH₃ | CH₃ | C(O)OCH₃ | C(O)OH | OCH(CH₃)₂ | H | NO₂ | H |
| DHP-591 | C₁₀H₂₁ | CH₃ | CH₃ | C(O)OCH₂CH₃ | C(O)OH | OCH(CH₃)₂ | H | NO₂ | H |
| DHP-592 | C₁₀H₂₁ | CH₃ | CH₃ | C(O)OCH(CH₃)₂ | C(O)OH | OCH(CH₃)₂ | H | NO₂ | H |
| DHP-593 | C₁₀H₂₁ | CH₃ | CH₃ | C(O)OCH₃ | C(O)OH | OCH₂CH₃ | H | NO₂ | H |
| DHP-594 | C₁₀H₂₁ | CH₃ | CH₃ | C(O)OCH₂CH₃ | C(O)OH | OCH₂CH₃ | H | NO₂ | H |
| DHP-595 | C₁₀H₂₁ | CH₃ | CH₃ | C(O)OCH(CH₃)₂ | C(O)OH | OCH₂CH₃ | H | NO₂ | H |
| DHP-596 | C₁₀H₂₁ | CH₃ | C# | C(O)OCH₂CH₃ | C(O)OCH₂CH₃ | OCH₃ | H | H | H |
| DHP-597 | C₁₀H₂₁ | CH₃ | C | C(O)OCH₂CH₃ | C(O)OCH₃ | OCH₃ | H | H | H |
| DHP-598 | C₁₀H₂₁ | CH₃ | C | C(O)OCH₂CH₃ | C(O)OCH(CH₃)₂ | OCH₃ | H | H | H |
| DHP-599 | C₁₀H₂₁ | CH₃ | C | C(O)OCH₂CH₃ | C(O)OCH₂CH₃ | OCH(CH₃)₂ | H | H | H |
| DHP-600 | C₁₀H₂₁ | CH₃ | C | C(O)OCH₂CH₃ | C(O)OCH₃ | OCH(CH₃)₂ | H | H | H |
| DHP-601 | C₁₀H₂₁ | CH₃ | C | C(O)OCH₂CH₃ | C(O)OCH(CH₃)₂ | OCH(CH₃)₂ | H | H | H |
| DHP-602 | C₁₀H₂₁ | CH₃ | C | C(O)OCH₂CH₃ | C(O)OCH₂CH₃ | OCH₂CH₃ | H | H | H |
| DHP-603 | C₁₀H₂₁ | CH₃ | C | C(O)OCH₂CH₃ | C(O)OCH₃ | OCH₂CH₃ | H | H | H |
| DHP-604 | C₁₀H₂₁ | CH₃ | C | C(O)OCH₂CH₃ | C(O)OCH(CH₃)₂ | OCH₂CH₃ | H | H | H |
| DHP-605 | C₁₀H₂₁ | CH₃ | C | C(O)OCH₃ | C(O)OCH₃ | OCH₃ | H | H | H |
| DHP-606 | C₁₀H₂₁ | CH₃ | C | C(O)OCH₃ | C(O)OCH₂CH₃ | OCH₃ | H | H | H |
| DHP-607 | C₁₀H₂₁ | CH₃ | C | C(O)OCH₃ | C(O)OCH(CH₃)₂ | OCH₃ | H | H | H |
| DHP-608 | C₁₀H₂₁ | CH₃ | C | C(O)OCH₃ | C(O)OCH₃ | OCH(CH₃)₂ | H | H | H |
| DHP-609 | C₁₀H₂₁ | CH₃ | C | C(O)OCH₃ | C(O)OCH₂CH₃ | OCH(CH₃)₂ | H | H | H |
| DHP-610 | C₁₀H₂₁ | CH₃ | C | C(O)OCH₃ | C(O)OCH(CH₃)₂ | OCH(CH₃)₂ | H | H | H |
| DHP-611 | C₁₀H₂₁ | CH₃ | C | C(O)OCH₃ | C(O)OCH₃ | OCH₂CH₃ | H | H | H |
| DHP-612 | C₁₀H₂₁ | CH₃ | C | C(O)OCH₃ | C(O)OCH₂CH₃ | OCH₂CH₃ | H | H | H |
| DHP-613 | C₁₀H₂₁ | CH₃ | C | C(O)OCH₃ | C(O)OCH(CH₃)₂ | OCH₂CH₃ | H | H | H |
| DHP-614 | C₁₀H₂₁ | CH₃ | C | C(O)OCH(CH₃)₂ | C(O)OCH(CH₃)₂ | OCH₃ | H | H | H |
| DHP-615 | C₁₀H₂₁ | CH₃ | C | C(O)OCH(CH₃)₂ | C(O)OCH₃ | OCH₃ | H | H | H |
| DHP-616 | C₁₀H₂₁ | CH₃ | C | C(O)OCH(CH₃)₂ | C(O)OCH₂CH₃ | OCH₃ | H | H | H |
| DHP-617 | C₁₀H₂₁ | CH₃ | C | C(O)OCH(CH₃)₂ | C(O)OCH(CH₃)₂ | OCH(CH₃)₂ | H | H | H |
| DHP-618 | C₁₀H₂₁ | CH₃ | C | C(O)OCH(CH₃)₂ | C(O)OCH₃ | OCH(CH₃)₂ | H | H | H |
| DHP-619 | C₁₀H₂₁ | CH₃ | C | C(O)OCH(CH₃)₂ | C(O)OCH₂CH₃ | OCH(CH₃)₂ | H | H | H |
| DHP-620 | C₁₀H₂₁ | CH₃ | C | C(O)OCH(CH₃)₂ | C(O)OCH(CH₃)₂ | OCH₂CH₃ | H | H | H |
| DHP-621 | C₁₀H₂₁ | CH₃ | C | C(O)OCH(CH₃)₂ | C(O)OCH₃ | OCH₂CH₃ | H | H | H |
| DHP-622 | C₁₀H₂₁ | CH₃ | C | C(O)OCH(CH₃)₂ | C(O)OCH₂CH₃ | OCH₂CH₃ | H | H | H |
| DHP-623 | C₁₀H₂₁ | CH₃ | C | C(O)OH | C(O)OH | OCH₃ | H | H | H |
| DHP-624 | C₁₀H₂₁ | CH₃ | C | C(O)OH | C(O)OCH₃ | OCH₃ | H | H | H |
| DHP-625 | C₁₀H₂₁ | CH₃ | C | C(O)OH | C(O)OCH₂CH₃ | OCH₃ | H | H | H |
| DHP-626 | C₁₀H₂₁ | CH₃ | C | C(O)OH | C(O)OCH(CH₃)₂ | OCH₃ | H | H | H |
| DHP-627 | C₁₀H₂₁ | CH₃ | C | C(O)OH | C(O)OH | OCH(CH₃)₂ | H | H | H |
| DHP-628 | C₁₀H₂₁ | CH₃ | C | C(O)OH | C(O)OCH₃ | OCH(CH₃)₂ | H | H | H |
| DHP-629 | C₁₀H₂₁ | CH₃ | C | C(O)OH | C(O)OCH₂CH₃ | OCH(CH₃)₂ | H | H | H |
| DHP-630 | C₁₀H₂₁ | CH₃ | C | C(O)OH | C(O)OCH(CH₃)₂ | OCH(CH₃)₂ | H | H | H |
| DHP-631 | C₁₀H₂₁ | CH₃ | C | C(O)OH | C(O)OH | OCH₂CH₃ | H | H | H |
| DHP-632 | C₁₀H₂₁ | CH₃ | C | C(O)OH | C(O)OCH₃ | OCH₂CH₃ | H | H | H |
| DHP-633 | C₁₀H₂₁ | CH₃ | C | C(O)OH | C(O)OCH₂CH₃ | OCH₂CH₃ | H | H | H |
| DHP-634 | C₁₀H₂₁ | CH₃ | C | C(O)OH | C(O)OCH(CH₃)₂ | OCH₂CH₃ | H | H | H |
| DHP-635 | C₁₀H₂₁ | CH₃ | C | C(O)OCH₃ | C(O)OH | OCH₃ | H | H | H |
| DHP-636 | C₁₀H₂₁ | CH₃ | C | C(O)OCH₂CH₃ | C(O)OH | OCH₃ | H | H | H |
| DHP-637 | C₁₀H₂₁ | CH₃ | C | C(O)OCH(CH₃)₂ | C(O)OH | OCH₃ | H | H | H |
| DHP-638 | C₁₀H₂₁ | CH₃ | C | C(O)OCH₃ | C(O)OH | OCH(CH₃)₂ | H | H | H |
| DHP-639 | C₁₀H₂₁ | CH₃ | C | C(O)OCH₂CH₃ | C(O)OH | OCH(CH₃)₂ | H | H | H |
| DHP-640 | C₁₀H₂₁ | CH₃ | C | C(O)OCH(CH₃)₂ | C(O)OH | OCH(CH₃)₂ | H | H | H |
| DHP-641 | C₁₀H₂₁ | CH₃ | C | C(O)OCH₃ | C(O)OH | OCH₂CH₃ | H | H | H |
| DHP-642 | C₁₀H₂₁ | CH₃ | C | C(O)OCH₂CH₃ | C(O)OH | OCH₂CH₃ | H | H | H |
| DHP-643 | C₁₀H₂₁ | CH₃ | C | C(O)OCH(CH₃)₂ | C(O)OH | OCH₂CH₃ | H | H | H |
| DHP-644 | C₁₀H₂₁ | CH₃ | CH₃ | CH₂OCH₃ | CH₂OCH₃ | OCH₃ | H | H | H |
| DHP-645 | C₁₀H₂₁ | CH₃ | CH₃ | CH₂OCH₃ | CH₂OCH₂CH₃ | OCH₃ | H | H | H |
| DHP-646 | C₁₀H₂₁ | CH₃ | CH₃ | CH₂OCH₃ | CH₂OCH(CH₃)₂ | OCH₃ | H | H | H |
| DHP-647 | C₁₀H₂₁ | CH₃ | CH₃ | CH₂OCH₂CH₃ | CH₂OCH₃ | OCH(CH₃)₂ | H | H | H |
| DHP-648 | C₁₀H₂₁ | CH₃ | CH₃ | CH₂OCH₂CH₃ | CH₂OCH₂CH₃ | OCH(CH₃)₂ | H | H | H |
| DHP-649 | C₁₀H₂₁ | CH₃ | CH₃ | CH₂OCH₂CH₃ | CH₂OCH(CH₃)₂ | OCH(CH₃)₂ | H | H | H |
| DHP-650 | C₁₀H₂₁ | CH₃ | CH₃ | CH₂OCH(CH₃)₂ | CH₂OCH₃ | OCH₂CH₃ | H | H | H |
| DHP-651 | C₁₀H₂₁ | CH₃ | CH₃ | CH₂OCH(CH₃)₂ | CH₂OCH₂CH₃ | OCH₂CH₃ | H | H | H |
| DHP-652 | C₁₀H₂₁ | CH₃ | CH₃ | CH₂OCH(CH₃)₂ | CH₂OCH(CH₃)₂ | OCH₂CH₃ | H | H | H |
| DHP-653 | C₁₅H₃₁ | CH₃ | CH₃ | C(O)OCH₂CH₂OCH₃ | C(O)OCH₂CH₃ | OCH₂CH₃ | H | H | H |
| DHP-654 | C₁₅H₃₁ | CH₃ | CH₃ | C(O)OCH₂CH₃ | C(O)OCH₂CH₃ | OCH₂CH₂OCH₃ | H | H | H |
| DHP-655 | C₁₅H₃₁ | CH₃ | CH₃ | C(O)OCH₂CH₃ | C(O)OCH₂CH₃ | OCH₂CH₃ | H | NH₂ | H |

In all of the above compounds, R₁₀=R₁₁=H.

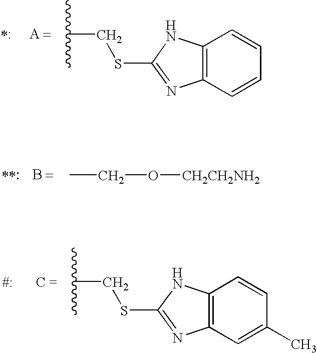

Also contemplated as part of the present invention are four other series of compounds resembling those in Table 1. These series are designated by the suffixes -11, -12, -13, and -14. They are identical to DHP-1 through DHP-326 and DHP-653 through DHP-655, except that in each of the -11 series compounds R₁ is C₁₁H₂₃; in each of the -12 series compounds R₁ is C₁₂H₂₅; in each of the -13 series compounds R₁ is C₁₃H₂₇; and in each of the -14 series compounds R₁ is C₁₄H₂₉. Thus, the inventors specifically contemplate each of DHP-1-11 through DHP-326-11 and DHP-653-11 through DHP-655-11; DHP-1-12 through DHP-326-12 and DHP-653-12 through DHP-655-12; DHP-1-13 through DHP-326-13 and DHP-653-13 through DHP-655-13; and DHP-1-14 through DHP-326-14 and DHP-653-14 through DHP-655-14, as explicitly as if the exact formula of each was set forth herein individually.

In another aspect, the invention relates to a method of treating cardiovascular disease or renal disease comprising identifying a patient in need of such treatment, and administering a pharmaceutical composition as described herein to said patient. In certain embodiments, the patient may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the patient is a human.

In some embodiments, the administering step comprises administering said ACE inhibitor or said ARB and said CCB nearly simultaneously. These embodiments include those in which the CCB and the ACE inhibitor or ARB are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments the administering step comprises administering one of the ACE inhibitor or ARB and the CCB first and then administering the other one of the ACE inhibitor or ARB and the CCB. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally.

The methods of the present invention are intended to provide treatment for cardiovascular disease, which may include congestive heart failure, hypertension, asymptomatic left ventricular dysfunction, or acute myocardial infarction. In some instances, patients suffering from a cardiovascular disease are in need of after-load reduction. The methods of the present invention are suitable to provide treatment for these patients as well.

The methods of the present invention are also intended to provide treatment for renal disease, which may include renal hypertrophy, renal hyperplasia, microproteinuria, proteinuria, diabetic nephropathy, contrast-mediated nephropathy, toxin-induced renal injury, or oxygen free-radical mediated nephropathyhypertensive nephropathy, diabetic nephropathy, contrast-mediated nephropathy, toxin-induced renal injury, or oxygen free-radical mediated nephropathy.

In another aspect, the invention relates to a pharmaceutical composition comprising a combination of an CCB and an ACE inhibitor or ARB, as described above, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18 th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl cellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacqucr solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient.

The daily dosage regimen for an adult human patient may be, for example, an oral dose of between 0.1 mg and 500 mg, preferably between 1 mg and 250 mg, e.g. 5 to 200 mg or an intravenous, subcutaneous, or intramuscular dose of between 0.01 mg and 100 mg, preferably between 0.1 mg and 60 mg, e.g. 1 to 40 mg of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A pharmaceutical composition comprising an angiotensin converting enzyme (ACE) inhibitor and a calcium channel blocker (CCB), wherein said CCB is a compound of Formula I or II 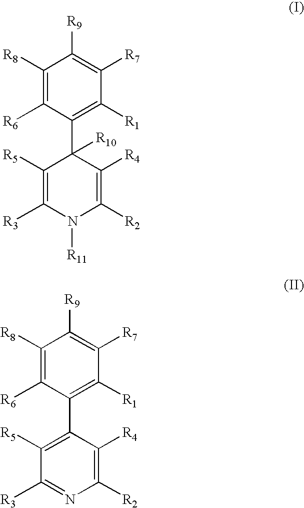
or a pharmaceutically acceptable salt, amide, ester, or prodrug thereof, where
a) R₁ is an straight-chain, branched, or cyclic alkyl group having greater than eight carbon atoms;
b) R₂-R₉ are each independently selected from the group consisting of hydrogen, halogen, perhaloalkyl, nitro, amino, a diazo salt, optionally substituted lower alkyl, optionally substituted lower alkylene, optionally substituted lower alkoxy, optionally substituted lower alkoxyalkyl, optionally substituted lower alkoxyalkoxy, optionally substituted lower mercaptyl, optionally substituted lower mercaptoalkyl, optionally substituted lower mercaptomercaptyl, —C(O)OH, —OC(O)H, —C(O)OR, —OC(O)R, —C(S)OR, —OC(S)R, —C(O)SR, —SC(O)R, —C(S)SR, —SC(S)R, C-amido, N-amido, and optionally substituted five- or six-membered heteroaryl ring or optionally substituted six-membered aryl or heteroaryl ring,
where the lower alkyl and the lower alkylene moieties are each independently and optionally substituted with one or more substituents selected from the group consisting of halogen, perhaloalkyl, nitro, amino, hydroxy, alkoxy, sulfhydryl, thioether, cyano, amido, ester, and 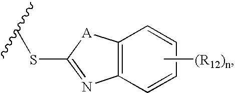
where A is selected from the group consisting of oxygen, sulfur, and —NH and R₁₂ is selected for the group consisting of hydrogen, hydroxy, alkoxy, haloalkoxy, halogen, haloalkyl, perhaloalkyl, nitro, amino, and a diazo salt, and n is between 0-4; and
where the ring moieties are each independently and optionally substituted with one or more substituents selected from the group consisting of lower alkyl, lower alkylene,
c) R₁₀ and R₁₁ in the compound of Formula I are each independently selected from the group consisting of hydrogen and lower alkyl.

2. A pharmaceutical composition comprising an angiotensin II receptor blocker (ARB) and a calcium channel blocker (CCB), wherein said CCB is a compound of Formula I or II 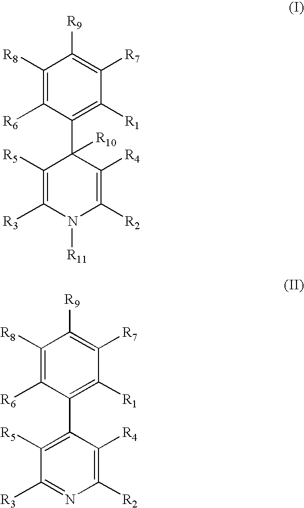
or a pharmaceutically acceptable salt, amide, ester, or prodrug thereof, where
a) R₁ is an straight-chain, branched, or cyclic alkyl group having greater than eight carbon atoms;
b) R₂-R₉ are each independently selected from the group consisting of hydrogen, halogen, perhaloalkyl, nitro, amino, a diazo salt, optionally substituted lower alkyl, optionally substituted lower alkylene, optionally substituted lower alkoxy, optionally substituted lower alkoxyalkyl, optionally substituted lower alkoxyalkoxy, optionally substituted lower mercaptyl, optionally substituted lower mercaptoalkyl, optionally substituted lower mercaptomercaptyl, —C(O)OH, —OC(O)H, —C(O)OR, —OC(O)R, —C(S)OR, —OC(S)R, —C(O)SR, —SC(O)R, —C(S)SR, —SC(S)R, C-amido, N-amido, and optionally substituted five- or six-membered heteroaryl ring or optionally substituted six-membered aryl or heteroaryl ring,
where the lower alkyl and the lower alkylene moieties are each independently and optionally substituted with one or more substituents selected from the group consisting of halogen, perhaloalkyl, nitro, amino, hydroxy, alkoxy, sulfhydryl, thioether, cyano, amido, ester, and 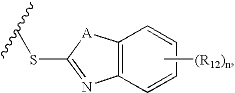
where A is selected from the group consisting of oxygen, sulfur, and —NH and R₁₂ is selected for the group consisting of hydrogen, hydroxy, alkoxy, haloalkoxy, halogen, haloalkyl, perhaloalkyl, nitro, amino, and a diazo salt, and n is between 0-4; and
where the ring moieties are each independently and optionally substituted with one or more substituents selected from the group consisting of lower alkyl, lower alkylene,
c) R₁₀ and R₁₁ in the compound of Formula I are each independently selected from the group consisting of hydrogen and lower alkyl.

3. A pharmaceutical composition comprising an angiotensin converting enzyme (ACE) inhibitor, an angiotensin II receptor blocker (ARB), and a calcium channel blocker (CCB), wherein said CCB is a compound of Formula I or II 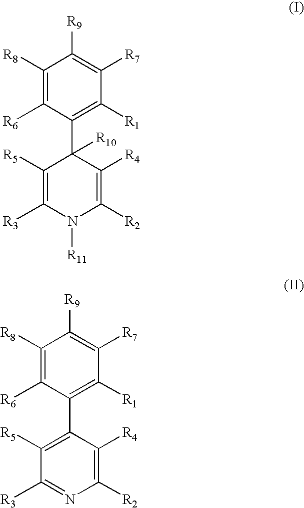
or a pharmaceutically acceptable salt, amide, ester, or prodrug thereof, where
a) R₁ is an straight-chain, branched, or cyclic alkyl group having greater than eight carbon atoms;
b) R₂-R₉ are each independently selected from the group consisting of hydrogen, halogen, perhaloalkyl, nitro, amino, a diazo salt, optionally substituted lower alkyl, optionally substituted lower alkylene, optionally substituted lower alkoxy, optionally substituted lower alkoxyalkyl, optionally substituted lower alkoxyalkoxy, optionally substituted lower mercaptyl, optionally substituted lower mercaptoalkyl, optionally substituted lower mercaptomercaptyl, —C(O)OH, —OC(O)H, —C(O)OR, —OC(O)R, —C(S)OR, —OC(S)R, —C(O)SR, —SC(O)R, —C(S)SR, —SC(S)R, C-amido, N-amido, and optionally substituted five- or six-membered heteroaryl ring or optionally substituted six-membered aryl or heteroaryl ring,
where the lower alkyl and the lower alkylene moieties are each independently and optionally substituted with one or more substituents selected from the group consisting of halogen, perhaloalkyl, nitro, amino, hydroxy, alkoxy, sulfhydryl, thioether, cyano, amido, ester, and 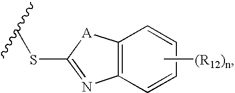
where A is selected from the group consisting of oxygen, sulfur, and —NH and R₁₂ is selected for the group consisting of hydrogen, hydroxy, alkoxy, haloalkoxy, halogen, haloalkyl, perhaloalkyl, nitro, amino, and a diazo salt, and n is between 0-4; and
where the ring moieties are each independently and optionally substituted with one or more substituents selected from the group consisting of lower alkyl, lower alkylene,
c) R₁₀ and R₁₁ in the compound of Formula I are each independently selected from the group consisting of hydrogen and lower alkyl.

4. The composition of claim 1, wherein said ACE inhibitor is selected from the group consisting of lisinopril, enalapril, quinapril, ramipril, benazepril, captopril, fosinopril, moexipril, trandolapril, and perindopril, or a pharmaceutically acceptable salt, prodrug, ester, or amide thereof.

5. The composition of claim 2, wherein said ARB is selected from the group consisting of losartan, irbesartan, candesartan, telmisartan, eposartan, and valsartan

6. The composition of claim 1, wherein R₄ is 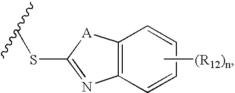
wherein A is selected from the group consisting of oxygen, sulfur, and —NH;
R₁₂ is selected from the group consisting of hydrogen, hydroxy, alkoxy, haloalkoxy, halogen, haloalkyl, perhaloalkyl, nitro, amino, and a diazo salt, and n is between 0-4.

7. The composition of claim 1, wherein R₄ and R₅ are each independently selected from the group consisting of
a) an optionally substituted alkyl group;
b) an alkoxy of formula —(X)ₙ₁—O—X₂, where
X₁ is selected from the group consisting of lower alkylene, lower alkenylene, lower alkynylene, aryl, and heteroaryl;
X₂ is selected from the group consisting of hydrogen, lower alkyl, aryl, and heteroaryl; and
n1 is 0 or 1; and
c) a thioether or thiol of formula —(X₃)ₙ₃—S—X₄, where
X₃ is selected from the group consisting of lower alkylene, lower alkenylene, lower alkynylene, aryl, and heteroaryl;
X₄ is selected from the group consisting of hydrogen, lower alkyl, aryl, and heteroaryl; and
n3 is 0 or 1;
d) a carboxylic acid of formula —(X₅)ₙ₅—C(=E)-E′H, where
X₅ is selected from the group consisting of lower alkylene, lower alkenylene, lower alkynylene, aryl, and heteroaryl;
E and E′ are each independently selected from the group consisting of oxygen and sulfur;
n5 is 0 or 1; and
e) an ester of formula —(X₆)ₙ₆—C(=E)-E′X₇, or of formula —(X₆)ₙ₆-E′-C(=E)-X₇, where
X₆ is selected from the group consisting of lower alkylene, lower alkenylene, lower alkynylene, aryl, and heteroaryl;
E and E′ are each independently selected from the group consisting of oxygen and sulfur;
X₇ is selected from the group consisting of hydrogen, lower alkyl, aryl, heteroaryl, hydroxy, alkoxy, amino, and —NX₈X₉,
where X₈ and X₉ are each independently selected from the group consisting of hydrogen, alkyl, aryl, and heteroaryl; and
n6 is 0 or 1.

8. The composition of claim 1, wherein R₄ and R₅ are each independently lower alkyl.

9. The composition of claim 1, wherein R₄ and R₅ are selected from the group consisting of methyl, ethyl, n-propyl, isopropyl, n-butyl, sec-butyl, and tert-butyl.

10. The composition of claim 1, wherein R₆ is selected from the group consisting of
a) hydrogen;
b) an optionally substituted alkyl group;
c) an alkoxy of formula —(X₁)ₙ₁—O—X₂, where
X₁ is selected from the group consisting of lower alkylene, lower alkenylene, lower alkynylene, aryl, and heteroaryl;
X₂ is selected from the group consisting of hydrogen, lower alkyl, aryl, and heteroaryl; and
n1 is 0 or 1; and
d) a thioether or thiol of formula —(X₃)ₙ₃—S—X₄, where
X₃ is selected from the group consisting of lower alkylene, lower alkenylene, lower alkynylene, aryl, and heteroaryl;
X₄ is selected from the group consisting of hydrogen, lower alkyl, aryl, and heteroaryl; and
n3 is 0 or 1;
e) a carboxylic acid of formula —(X₅)ₙ₅—C(=E)-E′H, where
X₅ is selected from the group consisting of lower alkylene, lower alkenylene, lower alkynylene, aryl, and heteroaryl;
E and E′ are each independently selected from the group consisting of oxygen and sulfur;
n5 is 0 or 1; and
f) an ester of formula —(X₆)ₙ₆—C(=E)-E′X₇, or of formula —(X₆)ₙ₆-E′-C(=E)-X₇, where
X₆ is selected from the group consisting of lower alkylene, lower alkenylene, lower alkynylene, aryl, and heteroaryl;
E and E′ are each independently selected from the group consisting of oxygen and sulfur;
X₇ is selected from the group consisting of hydrogen, lower alkyl, aryl, heteroaryl, hydroxy, alkoxy, amino, and —NX₈X₉,
where X₈ and X₉ are each independently selected from the group consisting of hydrogen, alkyl, aryl, and heteroaryl; and
n6 is 0 or 1.

11. The composition of claim 1, wherein R₇-R₉ are each independently selected from the group consisting of
a) hydrogen;
b) an optionally substituted alkyl group;
c) an alkoxy of formula —(X₁)ₙ₁—O—X₂, where
X₁ is selected from the group consisting of lower alkylene, lower alkenylene, lower alkynylene, aryl, and heteroaryl;
X₂ is selected from the group consisting of hydrogen, lower alkyl, aryl, and heteroaryl; and
n1 is 0 or 1; and
d) a thioether or thiol of formula —(X₃)ₙ₃—S—X₄, where
X₃ is selected from the group consisting of lower alkylene, lower alkenylene, lower alkynylene, aryl, and heteroaryl;
X₄ is selected from the group consisting of hydrogen, lower alkyl, aryl, and heteroaryl; and
n3 is 0 or 1;
e) a carboxylic acid of formula —(X₅)ₙ₅—C(=E)-E′H, where
X₅ is selected from the group consisting of lower alkylene, lower alkenylene, lower alkynylene, aryl, and heteroaryl;
E and E′ are each independently selected from the group consisting of oxygen and sulfur;
n5 is 0 or 1;
f) an ester of formula —(X₆)ₙ₆—C(=E)-E′X₇, or of formula —(X₆)ₙ₆-E′-C(=E)-X₇, where
X₆ is selected from the group consisting of lower alkylene, lower alkenylene, lower alkynylene, aryl, and heteroaryl;
E and E′ are each independently selected from the group consisting of oxygen and sulfur;
X₇ is selected from the group consisting of hydrogen, lower alkyl, aryl, heteroaryl, hydroxy, alkoxy, amino, and —NX₈X₉,
where X₈ and X₉ are each independently selected from the group consisting of hydrogen, alkyl, aryl, and heteroaryl; and
n6 is 0 or 1;
g) an amine of formula —(X₁₀)ₙ₁₀—NX₁₁X₁₂, where
X₁₀ is selected from the group consisting of lower alkylene, lower alkenylene, lower alkynylene, aryl, and heteroaryl;
where X₁₀ and X₁₁ are each independently selected from the group consisting of hydrogen, alkyl, aryl, and heteroaryl; and
n10 is 0 or 1;
h) NO₂;
i) halogen or perhaloalkyl; and
j) CN.

12. The composition of claim 11, wherein said alkyl is a lower alkyl.

13. The composition of claim 12, wherein said lower alkyl is selected from the group consisting of methyl, ethyl, and isopropyl.

14. The composition of claim 12, wherein R₇-R₉ are each independently selected from the group consisting of hydrogen, hydroxy, cyano (CN), nitro (NO₂), amino (NH₂), methyl, ethyl, isopropyl, fluoro, and chloro.

15. The composition of claim 12, wherein R₇-R₉ are the same.

16. The composition of claim 12, wherein R₇-R₉ are different.

17. The composition of claim 1, wherein R₁₀ and R₁₁ are each independently selected from the group consisting of hydrogen and alkyl.

18. The composition of claim 1, wherein said alkyl is a lower alkyl.

19. The composition of claim 18, wherein said lower alkyl is selected from the group consisting of methyl, ethyl, and isopropyl.

20. The composition of claim 1, wherein R₁₀ and R₁₁ are each hydrogen.

21. The composition claim 1, wherein said CCB is selected from the group consisting of
diethyl 1,4-dihydro-4-(2′-ethoxy-6′-pentadecylphenyl)-2,6-dimethyl-3,5-pyridine dicarboxylate,
dimethyl 1,4-dihydro-4-(2′-ethoxy-6′-pentadecylphenyl)-2,6-dimethyl-3,5-pyridine dicarboxylate,
diisopropyl 1,4-dihydro-4-(2′-ethoxy-6′-pentadecylphenyl)-2,6-dimethyl-3,5-pyridine dicarboxylate,
diethyl 1,4-dihydro-4-(2′-methoxy-6′-pentadecylphenyl)-2,6-dimethyl-3,5-pyridine dicarboxylate,
dimethyl 1,4-dihydro-4-(2′-methoxy-6′-pentadecylphenyl)-2,6-dimethyl-3,5-pyridine dicarboxylate,
diisopropyl 1,4-dihydro-4-(2′-methoxy-6′-pentadecylphenyl)-2,6-dimethyl-3,5-carboxy pyridine dicarboxylate,
diethyl 1,4-dihydro-4-(2′-isopropoxy-6′-pentadecylphenyl)-2,6-dimethyl-3,5-pyridine dicarboxylate,
dimethyl 1,4-dihydro-4-(2′-isopropoxy-6′-pentadecylphenyl)-2,6-dimethyl-3,5-pyridine dicarboxylate,
diisopropyl 1,4-dihydro-4-(2′-isopropoxy-6′-pentadecylphenyl)-2,6-dimethyl-3,5-pyridine dicarboxylate,
diethyl 1,4-dihydro-4-(2′-methoxy-6′-pentadecylphenyl)-2-methyl-6-(2′-mercapto-1′H-benzimidazolyl) methyl-3,5-pyridine dicarboxylate,
dimethyl 1,4-dihydro-4-(2′-methoxy-6′-pentadecylphenyl)-2-methyl-6-(2′-mercapto-1′H-benzimidazolyl) methyl-3,5-pyridine dicarboxylate,
diisopropyl 1,4-dihydro-4-(2′-methoxy-6′-pentadecylphenyl)-2-methyl-6-(2′-mercapto-1′H-benzimidazolyl) methyl-3,5-pyridine dicarboxylate,
diethyl 1,4-dihydro-4-(2′-isopropoxy-6′-pentadecylphenyl)-2-methyl-6-(2′-mercapto-1′H-benzimidazolyl) methyl-3,5-pyridine dicarboxylate,
dimethyl 1,4-dihydro-4-(2′-isopropoxy-6′-pentadecylphenyl)-2-methyl-6-(2′-mercapto-1′H-benzimidazolyl) methyl-3,5-pyridine dicarboxylate,
diisopropyl 1,4-dihydro-4-(2′-isopropoxy-6′-pentadecylphenyl)-2-methyl-6-(2′-mercapto-1′H-benzimidazolyl) methyl-3,5-pyridine dicarboxylate,
diethyl 1,4-dihydro-4-(2′-ethoxy-6′-pentadecylphenyl)-2-methyl-6-(2′-mercapto-1′H-benzimidazolyl) methyl-3,5-pyridine dicarboxylate,
dimethyl 1,4-dihydro-4-(2′-ethoxy-6′-pentadecylphenyl)-2-methyl-6-(2′-mercapto-1′H-benzimidazolyl) methyl-3,5-pyridine dicarboxylate,
diisopropyl 1,4-dihydro-4-(2′-ethoxy-6′-pentadecylphenyl)-2-methyl-6-(2′-mercapto-1′H-benzimidazolyl) methyl-3,5-pyridine dicarboxylate,
1,4-dihydro-4-(2′-ethoxy-6′-pentadecylphenyl)-2,6-dimethyl-3-ethyl-5-(methoxy ethyl) pyridine dicarboxylate,
1,4-dihydro-4-(2′-ethoxy-6′-pentadecylphenyl)-2,6-dimethyl-3-methyl-5-(methoxy ethyl) pyridine dicarboxylate,
1,4-dihydro-4-(2′-ethoxy-6′-pentadecylphenyl)-2,6-dimethyl-3-isopropyl-5-(methoxy ethyl) pyridine dicarboxylate,
1,4-dihydro-4-(2′-methoxy-6′-pentadecylphenyl)-2,6-dimethyl-3-ethyl-5-(methoxy ethyl) pyridine dicarboxylate,
1,4-dihydro-4-(2′-isopropoxy-6′-pentadecylphenyl)-2,6-dimethyl-3-ethyl-5-(methoxy ethyl) pyridine dicarboxylate,
diethyl 1,4-dihydro-4-(2′-ethoxy-6′-pentadecylphenyl)-2-(2′-amino ethoxy) methyl-6-methyl-3,5-pyridine dicarboxylate,
dimethyl 1,4-dihydro-4-(2′-ethoxy-6′-pentadecylphenyl)-2-(2′-amino ethoxy) methyl-6-methyl-3,5-pyridine dicarboxylate,
diisopropyl 1,4-dihydro-4-(2′-ethoxy-6′-pentadecylphenyl)-2-(2′-amino ethoxy) methyl-6-methyl-3,5-pyridine dicarboxylate,
diethyl 1,4-dihydro-4-(2′-methoxy-6′-pentadecylphenyl)-2-(2′-amino ethoxy) methyl-6-methyl-3,5-pyridine dicarboxylate,
dimethyl 1,4-dihydro-4-(2′-methoxy-6′-pentadecylphenyl)-2-(2′-amino ethoxy) methyl-6-methyl-3,5-pyridine dicarboxylate,
diisopropyl 1,4-dihydro-4-(2′-methoxy-6′-pentadecylphenyl)-2-(2′-amino ethoxy) methyl-6-methyl-3,5-pyridine dicarboxylate,
diethyl 1,4-dihydro-4-(2′-isopropoxy-6′-pentadecylphenyl)-2-((2′-amino ethoxy) methyl-6-methyl-3,5-pyridine dicarboxylate,
dimethyl 1,4-dihydro-4-(2′-isopropoxy-6′-pentadecylphenyl)-2-(2′-amino ethoxy) methyl-6-methyl-3,5-pyridine dicarboxylate,
diisopropyl 1,4-dihydro-4-(2′-isopropoxy-6′-pentadecylphenyl)-2-(2′-amino ethoxy) methyl-6-methyl-3,5-pyridine dicarboxylate,
diethyl 1,4-dihydro-4-(2′-ethoxy-3′,5′-dinitro-6′-pentadecylphenyl)-2,6-dimethyl-3,5-pyridine dicarboxylate,
dimethyl 1,4-dihydro-4-(2′-ethoxy-3′,5′-dinitro-6′-pentadecylphenyl)-2,6-dimethyl-3,5-pyridine dicarboxylate,
diisopropyl 1,4-dihydro-4-(2′-ethoxy-3′,5′-dinitro-6′-pentadecylphenyl)-2,6-dimethyl-3,5-pyridine dicarboxylate,
diethyl 1,4-dihydro-4-(2′-methoxy-3′,5′-dinitro-6′-pentadecylphenyl)-2,6-dimethyl-3,5-pyridine dicarboxylate,
dimethyl 1,4-dihydro-4-(2′-methoxy-3′,5′-dinitro-6′-pentadecylphenyl)-2,6-dimethyl-3,5-pyridine dicarboxylate,
diisopropyl 1,4-dihydro-4-(2′-methoxy-3′,5′-dinitro-6′-pentadecylphenyl)-2,6-dimethyl-3,5-pyridine dicarboxylate,
diethyl 1,4-dihydro-4-(2′-isopropoxy-3′,5′-dinitro-6′-pentadecylphenyl)-2,6-dimethyl-3,5-pyridine dicarboxylate,
dimethyl 1,4-dihydro-4-(2′-isopropoxy-3′,5′-dinitro-6′-pentadecylphenyl)-2,6-dimethyl-3,5-pyridine dicarboxylate,
diisopropyl 1,4-dihydro-4-(2′-isopropoxy-3′,5′-dinitro-6′-pentadecylphenyl)-2,6-dimethyl-3,5-pyridine dicarboxylate,
diethyl 1,4-dihydro-4-(2′-ethoxy-3′,5′-diamino-6′-pentadecylphenyl)-2,6-dimethyl-3,5-pyridine dicarboxylate,
dimethyl 1,4-dihydro-4-(2′-ethoxy-3′,5′-diamino-6′-pentadecylphenyl)-2,6-dimethyl-3,5-pyridine dicarboxylate,
diisopropyl 1,4-dihydro-4-(2′-ethoxy-3′,5′-diamino-6′-pentadecylphenyl)-2,6-dimethyl-3,5-pyridine dicarboxylate,
diethyl 1,4-dihydro-4-(2′-methoxy-3′,5′-diamino-6′-pentadecylphenyl)-2,6-dimethyl-3,5-pyridine dicarboxylate,
dimethyl 1,4-dihydro-4-(2′-methoxy-3′,5′-diamino-6′-pentadecylphenyl)-2,6-dimethyl-3,5-pyridine dicarboxylate,
diisopropyl 1,4-dihydro-4-(2′-methoxy-3′,5′-diamino-6′-pentadecylphenyl)-2,6-dimethyl-3,5-pyridine dicarboxylate,
diethyl 1,4-dihydro-4-(2′-isopropoxy-3′,5′-diamino-6′-pentadecylphenyl)-2,6-dimethyl-3,5-pyridine dicarboxylate,
dimethyl 1,4-dihydro-4-(2′-isopropoxy-3′,5′-diamino-6′-pentadecylphenyl)-2,6-dimethyl-3,5-pyridine dicarboxylate,
diisopropyl 1,4-dihydro-4-(2′-isopropoxy-3′,5′-diamino-6′-pentadecylphenyl)-2,6-dimethyl-3,5-pyridine dicarboxylate,
diethyl 1,4-dihydro-4-(2′-ethoxy-6′-pentadecylphenyl)-2-methyl-6-(5″-methyl-2-mercapto-1′H-benzimidazolyl)methyl-3,5-pyridine dicarboxylate,
dimethyl 1,4-dihydro-4-(2′-ethoxy-6′-pentadecylphenyl)-2-methyl-6-(5″-methyl-2-mercapto-1′H-benzimidazolyl)methyl-3,5-pyridine dicarboxylate,
diisopropyl 1,4-dihydro-4-(2′-ethoxy-6′-pentadecylphenyl)-2-methyl-6-(5″-methyl-2-mercapto-1′H-benzimidazolyl)methyl-3,5-pyridine dicarboxylate,
diethyl 1,4-dihydro-4-(2′-methoxy-6′-pentadecylphenyl)-2-methyl-6-(5″-methyl-2-mercapto-1′H-benzimidazolyl)methyl-3,5-pyridine dicarboxylate,
dimethyl 1,4-dihydro-4-(2′-methoxy-6′-pentadecylphenyl)-2-methyl-6-(5″-methyl-2-mercapto-1′H-benzimidazolyl)methyl-3,5-pyridine dicarboxylate,
diisopropyl 1,4-dihydro-4-(2′-methoxy-6′-pentadecylphenyl)-2-methyl-6-(5″-methyl-2-mercapto-1′H-benzimidazolyl)methyl-3,5-pyridine dicarboxylate,
diethyl 1,4-dihydro-4-(2′-isopropoxy-6′-pentadecylphenyl)-2-methyl-6-(5″-methyl-2-mercapto-1′H-benzimidazolyl)methyl-3,5-pyridine dicarboxylate,
dimethyl 1,4-dihydro-4-(2′-isopropoxy-6′-pentadecylphenyl)-2-methyl-6-methyl (5′-methyl-2-mercapto-1′H-benzimidazolyl)methyl-3,5-pyridine dicarboxylate, and
diisopropyl 1,4-dihydro-4-(2′-isopropoxy-6′-pentadecylphenyl)-2-methyl-6-methyl (5′-methyl-2-mercapto-1′H-benzimidazolyl)methyl-3,5-pyridine dicarboxylate.

22. The composition of claim 1, wherein said CCB is selected from the group consisting of DHP-1 through DHP-655, DHP-1-11 through DHP-326-11, DHP-653-11 through DHP-655-11, DHP-1-12 through DHP-326-12, DHP-653-12 through DHP-655-12, DHP-1-13 through DHP-326-13, DHP-653-13 through DHP-655-13, DHP-1-14 through DHP-326-14, and DHP-653-14 through DHP-655-14.

23. A method of treating cardiovascular disease or renal disease comprising identifying a patient in need of such treatment, and administering a pharmaceutical composition of claim 1 to said patient.

24. The method of claim 23, wherein said administering step comprises administering said ACE inhibitor and said CCB nearly simultaneously.

25. The method of claim 23, wherein said administering step comprises administering one of said ACE inhibitor and said CCB first and then administering the other one of said ACE inhibitor and said CCB.
